SEQUENCE LISTING (6 sequences)

SEQ ID NO: 1 - DNA, 1725 bp, Human respiratory syncytial virus S2
  CDS (1)..(1725)
atggagttgccaatcctcaaaacaaatgcaattaccgcaatccttgctgcagtcacactctgttttgcttccagtcaaaacatcactgaagaattttatcaatcaacatgcagtgcagtcagcaaaggctatcttagtgctctaagaactggttggtatactagtgttataactatagaattaagtaatatcaaggaaaataagtgtaatggaacagatgctaaggtaaaattgataaaacaagaattagataaatataaaagtgctgtaacagaattgcagttgctcatgcaaagcacaccggcaaccaacaatcgagccagaagagaactaccaaggtttatgaattatacactcaacaataccaaaaataccaatgtaacattaagcaagaaaaggaaaagaagatttcttggctttttgttaggtgttggatctgcaatcgccagtggcattgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacagttgttacctattgtgaacaagcaaagctgtagcatatcaaacattgaaactgtgatagagttccaacaaaagaacaacagactactagagattaccagggaatttagtgttaatgcaggtgtaactacacctgtaagcacttatatgttaacaaatagtgaattattatcattaatcaatgatatgcctataacaaatgatcagaaaaagttaatgtccaacaatgttcaaatagttagacagcaaagttactctatcatgtccataataaaggaggaagtcttagcatatgtagtacaattaccactatatggtgtaatagatacaccttgttggaaactacacacatcccctctatgtacaaccaacacaaaggaagggtccaacatctgtttaacaagaaccgacagaggatggtactgtgacaatgcaggatcagtatctttcttcccactagctgaaacatgtaaagttcaatcgaatcgagtattttgtgacacaatgaacagtttaacattaccaagtgaagtaaatctctgcaacattgacatattcaaccccaaatatgattgcaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataagggggtggacactgtgtctgtaggtaatacattatattatgtaaataagcaagaaggcaaaagtctctatgtaaaaggtgaaccaataataaatttctatgacccattagtgttcccctctgatgaatttgatgcatcaatatctcaagtcaatgagaagattaaccagagcctagcatttattcgtaaatccgatgaattattacataatgtaaatgctggtaaatccaccacaaatatcatgataactactataattatagtgattatagtaatattgttatcattaattgccgttggactgctcctatactgcaaggccagaagcacaccagtcacactaagcaaggatcaactgagtggtataaataatattgcatttagtaactaa

SEQ ID NO: 2 - protein, 574 aa, Human respiratory syncytial virus S2
MELPILKTNAITAILAAVTLCFASSQNITEEFYQSTCSAVSKGYLSALRTGWYTSVITIELSNIKENKCNGTDAKVKLIKQELDKYKSAVTELQLLMQSTPATNNRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 3 - DNA, 897 bp, Human respiratory syncytial virus S2
  CDS (1)..(897)
atgtccaaaaacaaggaccaacgcaccgccaagacactagaaaagacctgggacactctcaatcatctattattcatatcatcgtgcttatacaagttaaatcttaaatctatagcacaaatcacattatccattctggcaatgataatctcaacttcacttataattgcagccatcatattcatagcctcggcaaaccacaaagtcacactaacaactgcaatcatacaagatgcaacaagccagatcaagaacacaaccccaacatacctcacccagaatccccagcttggaatcagcttctccaatctgtctgaaactacatcacaaaccaccaccatactagcttcaacaacaccaagtgtcaagtcaaccctgcaatccacaacagtcaagaccaaaaacacaacaacaaccaaaatacaacccagcaagcccaccacaaaacaacgccaaaacaaaccaccaaacaaacccaataatgattttcactttgaagtgttcaactttgtaccttgcagcatatgcagcaacaatccaacctgctgggctatctgtaaaagaataccaaacaaaaaacctggaaagaaaaccaccaccaagcccacaaaaaaaccaaccatcaagacaaccaaaaaagatctcaaacctcaaaccacaaaaccaaaggaagtacctaccaccaagcccacagaaaagccaaccatcaacaccaccaaaacaaacatcagaactacactgctcaccaacaataccacaggaaatccagaacacacaagtcaaaagggaaccctccactcaacctcctccgatggcaatccaagcccttcacaagtctatacaacatccgagtacctatcacaacctccatctccatccaacacaacaaaccagtag

SEQ ID NO: 4 - protein, 298 aa, Human respiratory syncytial virus S2
MSKNKDQRTAKTLEKTWDTLNHLLFISSCLYKLNLKSIAQITLSILAMIISTSLIIAAIIFIASANHKVTLTTAIIQDATSQIKNTTPTYLTQNPQLGISFSNLSETTSQTTTILASTTPSVKSTLQSTTVKTKNTTTTKIQPSKPTTKQRQNKPPNKPNNDFHFEVFNFVPCSICSNNPTCWAICKRIPNKKPGKKTTTKPTKKPTIKTTKKDLKPQTTKPKEVPTTKPTEKPTINTTKTNIRTTLLTNNTTGNPEHTSQKGTLHSTSSDGNPSPSQVYTTSEYLSQPPSPSNTTNQ

SEQ ID NO: 5 - DNA, 1620 bp, Human metapneumovirus (HMPV)
  CDS (1)..(1620)
atgtcttggaaagtggtgatcattttttcattgctaataacacctcaacacggtcttaaagagagctacctagaagaatcatgtagcactataactgagggatatcttagtgttctgaggacaggttggtataccaacgtttttacattagaggtgggtgatgtagaaaaccttacatgttctgatggacctagcctaataaaaacagaattagatctgaccaaaagtgcactaagagagctcaaaacagtctctgctgaccaattggcaagagaggaacaaattgagaatcccagacaatctaggtttgttctaggagcaatagcactcggtgttgcaacagcagctgcagtcacagcaggtgttgcaattgccaaaaccatccggcttgagagtgaagtcacagcaattaagaatgccctcaaaacgaccaatgaagcagtatctacattggggaatggagttcgagtgttggcaactgcagtgagagagctgaaagactttgtgagcaagaatttaactcgtgcaatcaacaaaaacaagtgcgacattgatgacctaaaaatggccgttagcttcagtcaattcaacagaaggtttctaaatgttgtgcggcaattttcagacaatgctggaataacaccagcaatatctttggacttaatgacagatgctgaactagccagggccgtttctaacatgccgacatctgcaggacaaataaaattgatgttggagaaccgtgcgatggtgcgaagaaaggggttcggaatcctgataggggtctacgggagctccgtaatttacatggtgcagctgccaatctttggcgttatagacacgccttgctggatagtaaaagcagccccttcttgttccggaaaaaagggaaactatgcttgcctcttaagagaagaccaagggtggtattgtcagaatgcagggtcaactgtttactacccaaatgagaaagactgtgaaacaagaggagaccatgtcttttgcgacacagcagcgggaattaatgttgctgagcaatcaaaggagtgcaacatcaacatatccactacaaattacccatgcaaagtcagcacaggaagacatcctatcagtatggttgcactgtctcctcttggggctctggttgcttgctacaaaggagtaagctgttccattggcagcaacagagtagggatcatcaagcagctgaacaagggttgctcctatataaccaaccaagatgcagacacagtgacaatagacaacactgtatatcagctaagcaaagttgagggtgaacagcatgttataaaaggcagaccagtgtcaagcagctttgatccaatcaagtttcctgaagatcaattcaatgttgcacttgaccaagtttttgagaacattgaaaacagccaggccttggtagatcaatcaaacagaatcctaagcagtgcagagaaagggaatactggcttcatcattgtaataattctaattgctgtccttggctctagcatgatcctagtgagcatcttcattataatcaagaaaacaaagaaaccaacgggagcacctccagagctgagtggtgtcacaaacaatggcttcataccacacagttag

SEQ ID NO: 6 - protein, 539 aa, Human metapneumovirus (HMPV)
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHS

(19) United States
(12) Patent Application Publication
(10) Pub. No.: US 2009/0162395 A1
(43) Pub. Date: Jun. 25, 2009

(54) VACCINE FOR RSV AND MPV

(76) Inventors: James E. Crowe, JR., Nashville, TN (US); Hoyin Mok, Redwood City, CA (US); Robert E. Johnston, Chapel Hill, NC (US); John V. Williams, Brentwood, TN (US); Nancy L. Davis, Chapel Hill, NC (US)

Correspondence Address: FULBRIGHT & JAWORSKI L.L.P., 600 CONGRESS AVE., SUITE 2400, AUSTIN, TX, 78701, US

(21) Appl. No.: 12/238,130

(22) Filed: Sep. 25, 2008

Related U.S. Application Data
(60) Provisional application No. 60/975,431, filed on Sep. 26, 2007.

(51) Int. Cl. A61K 39/155; C12N 7/00

(52) U.S. Cl. 424/199.1; 435/235.1

(57) ABSTRACT

The present invention is directed to alphavirus vectored vaccine contructs encoding paramyxovirus proteins that find use in the prevention of respiratory syncytial virus or human metapneumovirus infections. In particular, these vaccines induce cellular and humoral immune responses that inhibit RSV. Also disclosed are improved methods for producing alphavirus vectored paramyxovirus vaccines.

This application claims benefit of priority to U.S. Provisional Application Ser. No. 60/975,431, filed Sep. 26, 2007, the entire contents of which are hereby incorporated by reference.

This invention was made with government support under grant number R01 AI-59597 awarded by the National Institutes of Allergy and Infectious Disease and the National Institutes of Health. The government has certain rights in the invention.

### BACKGROUND OF THE INVENTION

1. Field of the Invention

The present invention relates generally to the fields of molecular biology, genetics and virology. More particularly, it concerns the use of VEE replicions as vectors to deliver RSV and hMPV antigens to a host for the purpose of generating an immune response. Vaccines and methods of protecting a subject from RSV and hMPV infection also are provided.

2. Description of Related Art

Respiratory syncytial virus (RSV) is a paramyoxvirus that causes serious lower respiratory tract illness in infants and the elderly, making it a significant human pathogen. Significant morbidity and mortality for RSV is especially common in certain high-risk pediatric populations such as premature infants and infants with congenital heart or lung disorders. RSV bronchiolitis in infants is associated with recurrent wheezing and asthma later in childhood (Peebles, 2004; You et al., 2006). There are currently no FDA-approved vaccines for prevention of RSV disease by active immunization. Immunoprophylaxis by passive transfer of a humanized murine RSV fusion (F) protein-specific antibody is licensed for much of the high-risk infant population, but is not cost effective in otherwise healthy infants, who represent approximately 90% of those hospitalized with RSV.

Previous attempts to develop RSV vaccines have faced significant obstacles. An experimental formalin-inactivated RSV vaccine in the 1960s induced exacerbated disease and death in some vaccinated children during subsequent natural infection. It was shown subsequently that the formalin-inactivated RSV vaccine induced serum antibodies with poor neutralizing activity in infants (Murphy et al., 1986) and an atypical Th2-biased T cell response associated with enhanced histopathology following experimental immunization in small animals (Prince et al., 1986; Vaux-Peretz and Meignier, 1990). Treating RSV antigens with formaldehyde modifies the protein with carbonyl groups, which induce Th2-type responses preferentially and lead to enhanced disease (Moghaddam et al., 2006). Other attempts to generate RSV vaccines include using live-attenuated cold-adapted, temperature-sensitive mutant stains of RSV (Connors et al., 1995; Crowe et al., 1994a; Crowe et al., 1996a; Crowe et al., 1994b; Crowe et al., 1995; Crowe et al., 1993; Crowe et al., 1996b; Crowe et al., 1998; Firestone et al., 1996; Hsu et al., 1995; Juhasz et al., 1997; Karron et al., 1997; Karron et al., 2005), protein subunit vaccines coupled with adjuvant (Power et al., 1997; Welliver et al., 1994; Walsh, 1993; Homa et al., 1993) and RSV proteins expressed from recombinant viral vectors including vaccinia virus (Olmsted et al., 1986; Wyatt et al., 1999), adenovirus (Hsu et al., 1992), vesicular stomatitis virus (Kahn et al., 2001), Semliki Forest virus (Chen et al., 2002), bovine/human parainfluenza type 3 (Haller et al., 2003), Sendai virus (Takimoto et al., 2004) and Newcastle disease virus (Martinez-Sobrido et al., 2006).

The two surface glycoproteins of RSV, fusion (F) protein and attachment (G) protein, are the major antigenic targets for neutralizing antibodies. Neutralizing antibodies are sufficient to protect the lower respiratory tract (Connors et al., 1991). F and G proteins, therefore, have been used separately or in combination in many experimental RSV vaccines. Immunization with purified F protein alone or F protein expressed from a recombinant viral vector such as vaccinia virus induces RSV-specific neutralizing antibodies, CD8+ cytotoxic T lymphocytes and protection against subsequent RSV challenge in mice or cotton rats (Olmsted et al., 1986). Vaccination with G protein alone, however, often induces only partial protection against RSV challenge. In mice, the immune response against G is associated with eosinophilia and the induction of T_{H}2 type CD4+ lymphocytes in some experiments (Tebbey et al., 1998; Johnson et al., 1998; Hancock et al., 1996).

Human metapneumovirus (hMPV) is a paramyxovirus recently discovered in young children with respiratory tract disease (van den Hoogen et al., 2001). Subsequent studies show that hMPV is a causative agent for both upper and lower respiratory tracts infections in infants and young children (Boivin et al., 2002; Esper et al., 2004; Falsey et al., 2003; Williams et al., 2005; Williams et al., 2004). The spectrum of clinical illness ranges from cough and wheezing to bronchiolitis and pneumonia, similar to those seen in respiratory syncytial virus (RSV) and parainfluenza virus (PIV) infections. Children and adults with comorbid conditions, such as those with congenital heart and lung diseases, cancer and immunodeficiency, are particular at risk for acute respiratory disease from hMPV infection (Pelletier et al., 2002; Williams et al., 2005). Epidemiology studies, although not completely defined, has put HMPV infection incidence rate at 5-15% in young children (Boivin et al., 2002; Falsey et al., 2003; Williams and Harris, 2004; Pelletier et al., 2002; McAdam et al., 2004; Osterhaus and Fouchier, 2003). Recurrent infection of hMPV has also been documented (Ebihara et al., 2004). This, in combination with RSV and PIV, represents the leading causes for acute viral respiratory tract infections in this population and warrants the development of vaccine against this recently discovered virus.

Similarly to RSV, fusion F and attachment G proteins are the major surface glycoproteins on hMPV. Genetic analysis put hMPV into two subgroups (A and B) based on sequence comparison of these two genes in various clinical isolates (Bastien et al., 2003; Biacchesi et al., 2003). The subgroups are further divided into sublineages A1, A2, B1 and B2. The percent amino acid homology in the F protein reaches >95% and is highly conserved between the subgroups (Boivin et al., 2004; Skiadopoulos et al., 2004). G protein, however, shows significant amino acid diversification with homology ranging from 34-100% depending on inter- or intra-subgroup comparisons (Biacchesi et al., 2003; Bastien et al., 2004). In RSV, F and G proteins are the major antigenic targets for neutralizing antibodies. High titers of serum neutralizing antibodies are sufficient to protect the lower respiratory tract for RSV infection (Connors et al., 1991). Therefore, F and G proteins had been used singly or in combinations in various experimental vaccines.

As with RSV, a number of vaccines have been developed for hMPV. These include subunit F vaccine (Cseke et al., 2007), live-attenuated hMPV with gene deletions (Biacchesi et al., 2004) and a chimeric, live-attenuated PIV vaccine that incorporates the hMPV F, G or SH gene (Skiadopoulos et al., 2006; Tang et al., 2005; Tang et al., 2003). Although proven to be immunogenic in animal models, there are significant hurdles for some of these vaccines to be used in very young infants, which is one of the principle targets of hMPV vaccines. The presence of circulating maternal antibodies against most of the candidate vaccines and viral vectors is of concern and may blunt the efficacies of these vaccines in vivo. Furthermore, the ability to generate a mucosal response is pertinent to successful immunization against respiratory viruses.

Thus, a key determinant for optimal vaccination against respiratory viruses, such as RSV and human metapneumovirus (hMPV), is the ability of the vaccine to generate mucosal immunity. This goal can be achieved by using a topical route for vaccination or possibly by use of a vaccine construct that preferentially induces mucosal responses. Protection in the upper respiratory tract usually results only from immunization by the intranasal route, which can result in the induction of virus-specific mucosal IgA antibodies. However, as of yet a successful vaccine against viruses like RSV and hMPV has yet to be achieved.

### SUMMARY OF THE INVENTION

The invention comprises the use of alphavirus-vector constructs that generate virus replicon particles (VRPs) encoding the human metapneumovirus fusion or attachment proteins for active immunization against human metapneumovirus infection, and the use of such VRPs encoding the hRSV virus fusion or attachment proteins and hMPV fusion protein for active immunization against human respiratory syncytial virus infection.

Thus, in a particular embodiment, there is provided a virus replicon comprising (a) a Venezuelan equine encephalitis virus (VEE) positive-sense RNA genome lacking at least one functional gene for an VEE structural gene; and (b) a paramyxovirus surface glycoprotein coding region under the control of a promoter active in eukaryotic cells. The paramyoxovirus surface glycoprotein coding region may be from respiratory syncytial virus, such as RSV F or G, or from human metapneumovirus (hMPV), such as hMPV F. The promoter may be the VEE subgenomic 26S promoter, and the VEE RNA genome may be from pVR21. The VEE RNA genome may contain one more inactivating point mutations in one or more structural genes. The VEE RNA genome also may contain a truncating mutation in a structural gene or a deletion mutation in a structural gene.

In another embodiment, there is provided a method of inducing an immune response in an animal comprising administering to said animal an infectious virus particle comprising a viral replicon comprising (a) a Venezuelan equine encephalitis virus (VEE) positive-sense RNA genome lacking at least one functional gene for an VEE structural gene; and (b) a paramyxovirus surface glycoprotein coding region under the control of a promoter active in eukaryotic cells. The paramyoxovirus surface glycoprotein coding region may be from respiratory syncytial virus, such as RSV F or G, or from human metapneumovirus (hMPV), such as hMPV F. The promoter may be the VEE subgenomic 26S promoter, and the VEE RNA genome may be from pVR21. The VEE RNA genome may contain one more inactivating point mutations in one or more structural genes. The VEE RNA genome also may contain a truncating mutation in a structural gene or a deletion mutation in a structural gene.

Administration may comprise intranasal inhalation, subcutaneous injection or intramuscular injection. The method may further comprise administering said infectious virus particle a second time. The method may also further comprise administering said infectious virus particle a third time. The method may also further comprise assessing an immune response to said paramyxovirus surface glycoprotein, such as by RIA, ELISA, immunohistochemistry or Western blot. The animal may be a human or a mouse. The human may be a neonate comprising maternal antibodies. The immune response in said animal may be a humoral response, such as a mucosal IgA response, or a serum IgG response. The serum IgG response may be neutralizing. The immune response may be cellular, such as a balanced Th1/Th2 response.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Construction of Venezuelan Equine Encephalitis (VEE) transfer vector. RSV fusion protein (RSV.F) and RSV attachment protein (RSV.G) open reading frames were cloned into the VEE transfer vector, pVR21 via several steps. First, the VEE subgenomic 26S promoter was PCR amplified from pVR21 to generate amplicons that include the 26S leader mRNA sequence on the 3′ end. Secondly, RSV F or G amplicons were generated with a 26S leader mRNA sequence on the 5′ end. The two amplicons then were amplified to generate overlapping PCR products that contain RSV F or G genes under the control of the VEE subgenomic 26S promoter. Finally, the spliced PCR products were cloned back into pVR21 using unique restriction enzyme sites, SwaI and PacI, to produce pVR21-RSV.F or pVR21-RSV.G. Numbers in circles denote primers used in each PCR reaction.

FIGS. 2A-E. Infection of BHK-21 cells with VEE replicon particles encoding RSV.F (VRP-RSV.F) or RSV.G (VRP-RSV.G) leads to robust protein expression. Baby hamster kidney cells were infected at a moi of 5 with VRP-RSV.F or VRP-RSV.G. After 24 hours, immunostaining was performed on (FIG. 2A) uninfected or (FIG. 2B) VRP-RSV.F-infected BHK-21 cells with RSV F-specific mouse monoclonal antibodies. Secondary AlexaFluor C555-conjugated goat anti-mouse antibodies were used for fluorescence labeling. White arrow indicates fusion of multiple cells. Similar staining was performed with (FIG. 2C) uninfected or (FIG. 2D) VRP-RSV.G infected BHK cells with RSV G-specific mouse monoclonal antibodies. (FIG. 2E) In addition, Western blot was used to detect the presence of RSV F or G proteins in VRP infected BHK-21 cell lysates. The blot was probed with the same mouse monoclonal antibodies. Black arrows indicate the predicted apparent molecular weights of the proteins. Un-infected or RSV-infected cell lysates were used as negative or positive controls respectively.

FIGS. 3A-D. VRP-RSV.F induces RSV-F specific antibodies in the serum and mucosal secretions of VRP-vaccinated mice. BALB/c mice were vaccinated intranasally with 10⁶ infectious units of VRP-RSV.F on day 0 and 14. (FIG. 3A) Sera from vaccinated mice were obtained 28 days post vaccination. RSV-F specific enzyme-linked immunosorbent assay (ELISA) was performed on the sera with HRP-conjugated anti-mouse IgG antibodies. Amount of binding was determined from absorbance of HRP-substrate at λ=450 nm. (FIG. 3B) Nasal washes and (FIG. 3C) bronchioalveolar lavage (BAL) fluids also were obtained from vaccinated mice. The amounts of F-specific IgA antibodies were quantified similarly with HRP-conjugated anti-mouse IgA antibodies in an ELISA. ^{†}Data are for 3 out of 5 animals that responded. 2 animals did not make a detectable F-specific IgA response. (FIG. 3D) Sera from VRP-RSV.F vaccinated mice were isotyped for F-specific IgG1 and IgG2a antibodies. The ratios of IgG1 versus IgG2a were compared with sera from BALB/c or STAT-1 deficient mice infected with 10⁶ PFU of RSV A2. Each group in these experiments consisted of 5 animals.

FIG. 4. VRP-RSV.F induced equal or higher titers of RSV neutralizing antibodies in vaccinated mice than in animals infected with RSV or those vaccinated with VRP-RSV.G. Naïve BALB/c mice were immunized intranasally with increasing doses of VRP-RSV.F (10⁴, 10⁵ or 10⁶ IU) or VRP-RSV.G (10⁴ or 10⁶ IU) on day 0 and 14. Sera from vaccinated mice were tested for RSV neutralizing activity via a plaque reduction assay. Neutralizing activity is expressed as the geometric mean titer (GMT) of sera that neutralized 60% of plaques on RSV-infected HEp-2 cells. LLD indicates lower limit of detection.

FIGS. 5A-D. Two immunizations were sufficient to generate a maximal serum neutralizing antibodies response. BALB/c mice were vaccinated intranasally with VRP every 14 days for a total of 3 inoculations, as indicated by arrows. Sera were obtained every two weeks and neutralizing activities against RSV were measured. Values represent the geometric mean titer of 5 animals.

FIGS. 6A-D. RSV-F specific lymphocytes and splenocytes were induced in the lungs and spleens of mice immunized intranasally with VRPs. Lymphocytes and splenocytes were harvested from the lungs (FIGS. 6A and 6C) or spleens (FIGS. 6B and 6D) 7 days after vaccination. 2×10⁵ cells were stimulated with RSV F (aa. 85-93) peptides (FIGS. 6A and 6B) or RSV G (aa. 183-197) peptides (FIGS. 6C and 6D) in vitro for 20 hours and the numbers of IFN-γ spot forming cells were quantified by an ELISPOT assay. Spots were counted with an automated counting device and are expressed as numbers of spots per 10⁶ cells. Each experimental group contained 5 animals.

FIG. 7. IFN-γ gene expression levels 4 days after RSV challenge in the lungs of vaccinated BALB/c mice. IFN-γ gene expression levels were measured in lung lysates with real time PCR and expressed as the mean-fold change compared to uninfected control.

FIGS. 8A-D. Expression of hMPV proteins from VRP-infected BHK cells. BHK cells were either mock-infected (FIGS. 8A, 8C), infected at a moi of 5 with VRP-MPV.F (FIG. 8B) or infected at a moi of 5 with VRP-MPV.G (FIG. 8D). Cells then were fixed after 18 hours and immunostained for hMPV F (FIGS. 8A, 8B) or hMPV G (FIGS. 8C, 8D) protein expression using guinea pig polyclonal anti-hMPV antibodies.

FIGS. 9A-B. VRP-MPV.F induced hMPV-F or hMPV-G specific antibodies in the mucosal secretions of VRP-vaccinated mice. DBA/2 mice were vaccinated intranasally with 10⁶ infectious units of VRP-MPV.F or VRP-MPV.G on day 0 and 14. Nasal washes (FIG. 9A) or broncioalveolar lavage (BAL) fluids (FIG. 9B) were obtained from vaccinated mice 28 days post-vaccination. MPV-F or MPV-G specific enzyme-linked immunosorbent assay (ELISA) was performed on the samples with HRP-conjugated anti-mouse IgA antibodies. Amount of binding was determined from absorbance of HRP-substrate at λ=450 nm.

### DESCRIPTION OF ILLUSTRATIVE EMBODIMENTS

### I. The Present Invention

The inventors have developed VEE replicon particles as vectors to deliver RSV and hMPV surface glycoproteins and showed that these vaccine candidates induced immune responses comparable to or greater than those following wild-type virus infection. VEE replicons particles are attractive vaccine vectors for several reasons. First, they are less sensitive than most live viruses to type I interferons (White et al., 2001), which allows enhanced protein expression in replicon-infected cells in the draining lymph nodes. Translation of gene inserts from other alphaviruses, such as Sindbis virus, could be inhibited by such interferons (Ryman et al., 2005). Second, parenteral or intradermal inoculation of VEE replicons induces mucosal responses directed toward the encoded antigens. Most importantly, VRPs target specialized antigen presenting cells such as Langerhans cells in the dermis and human monocyte-derived dendritic cells (DCs) (Macdonald and Johnston, 2000; Moran et al., 2005). Compared to VEE replicons, other alphavirus vectors are not as effective in infecting DCs. Sindbis virus does target DCs but protein expression is shut down rapidly by the innate immune response (Ryman et al., 2005), and Semliki Forest virus does not infect DCs efficiently (Huckriede et al., 2004).

Expression of RSV and hMPV proteins from VRPs appeared authentic in every aspect. The inventors have incorporated the genes for RSV fusion (F) and attachment (G) glycoproteins into the replicons. F and G surface glycoproteins have been the targets for multiple experimental vaccines since these proteins are the targets for RSV neutralizing antibodies. In baby hamster kidney cells, VEE replicons expressed robust amounts of the encoded antigens. These antigens were expressed in a membrane-bound manner, which is consistent with published data in the distribution of F or G during RSV infection. When inoculated intranasally in mice and cotton rats, VEE replicons induced RSV-specific binding and neutralizing antibodies in both the systemic and mucosal immune compartments. Inoculation of VRPs via a mucosal site, the inventors observed a robust response against RSV in the respiratory tract and induced high levels of systemic RSV neutralizing antibodies. The RSV serum neutralizing titers induced by VRPs were directly proportional to vaccine dose, presumably due to increased in antigen expression from higher numbers of VRPs. Remarkably, the serum neutralizing titers of VRP-RSV.F vaccinated mice were higher than those following RSV infection, which demonstrates the potential of this vaccine. Mucosal IgA antibodies also were detected in the upper and lower respiratory tracts of vaccinated animals.

Vaccination with VRP encoding RSV F protein also induced F-specific CD8+ T lymphocytes. Upon stimulation with H-2K^{d} MHC class I restricted F epitopes, lung lymphocytes or splenocytes from VRP-RSV.F vaccinated mice secreted interferon-γ. RSV-specific cytotoxic T lymphocytes have been shown previously to contribute to resolution of infection and short-term protection against re-infection (Connors et al., 1992; Kulkarni et al., 1993). In contrast, VRP-RSV.G replicons induced much lower humoral and cellular immune responses in comparison to those responses induced by VRP-RSV.F. This finding could be caused by several factors, such as the expression level of G in vivo, the greater amount of glycosylation of G compared to F, and the need for complex processing of RSV G in vivo. Previous studies have revealed that RSV G is less immunogenic than RSV F.

A homologous prime-boost strategy was used to evaluate the efficacy of VRPs in inducing neutralizing antibodies at various time points post immunization. The inventors found that a single prime-boost was sufficient to induce a maximal level of neutralizing antibody responses. Further boosting with the same vectors had no effect in raising the neutralizing titer. When mice were challenged with RSV, only those that were vaccinated with VRP-RSV.F were protected completely in both the lungs and nasal turbinates. VRP-RSV.G vaccinated mice did not exhibit significant rises in neutralizing antibody titer, yet they were still protected in the lungs against RSV challenge. These mice may have produced low levels of neutralizing antibodies that could not be detected. In a semi-permissive small animal model, such immune responses may be sufficient to restrict RSV in vivo, however this level of immunogenicity is not likely to be effective in human subjects. RSV titers in the nasal turbinates of VRP-RSV.G vaccinated mice remained high. This finding is consistent with the low levels of antibodies and lack of antigen-specific CD4+/CD8+ T cells, which had been shown to correlate with upper respiratory tract protection in RSV-infected mice.

One of the major hurdles to development of a RSV vaccine is concern over safety in RSV-naïve recipients. Increased mortality rates and exacerbated diseases were seen in infants vaccinated with formalin-inactivated RSV in the 1960s during subsequent natural infection (Kapikian et al., 1969; Kim et al., 1969). Enhanced histopathology with excessive cellular influx and skewed Th2-dominant cytokine production were seen in animals vaccinated with formalin-inactivated RSV following viral challenge (Prince et al., 1986; Waris et al., 1996). The inventors performed multiple experiments to elucidate the types of responses in VRP-vaccinated mice pre- and post-challenge. The subclass distribution of antigen specific serum IgG1 was compared to IgG2a after immunization to evaluate the balance of Th1 versus Th2 responses. Mice immunized with VRP-RSV.F showed a balanced IgG1:IgG2a ratio (˜0.7) compared to RSV-infected STAT-1 deficient mice genetically predisposed to Th2 responses upon RSV infection (˜3.7). In addition, the inventors evaluated lung histopathology and cytokine gene expression in VRP-vaccinated mice after live RSV challenge. There was no evidence of enhanced lung histopathology in VRP-vaccinated animals upon RSV challenge, with minor peribronchiolar infiltrates and no significant airway mucus production. Unvaccinated animals did show minor increases in lung inflammation with peribronchiolar lymphocyte infiltration with a histopathology score similar to the immunized groups. The extent of inflammation in the lungs of these animals was not as dramatic as in some previous studies probably due to the fact that the doses of RSV inoculated and the A2 strain of RSV used differed from that of some previous studies.

Cytokine gene expression also was determined from lungs of these animals. Surprisingly, only IFN-γ gene expression was increased among all the cytokine genes tested. Infected groups had higher IFN-γ gene expression compared to uninfected controls. Interestingly, animals that had been vaccinated with VRP-RSV.F or VRP-RSV.G and those that were infected previously with RSV showed a dramatic increase in IFN-γ expression (˜3-12 times greater depending on the groups) over groups that were not previously vaccinated or that were vaccinated with an irrelevant VRP (VRP-MPV.F). This finding further suggests the development of properly balanced cellular immune responses in vaccinated animals upon RSV exposure. These results demonstrate that VEE replicon particles encoding RSV F protein induced strong antigen-specific humoral and cellular responses on mucosal surfaces and protected animals against intranasal RSV challenge.

The inventors have also demonstrated that VEE replicon particles encoding human metapneumovirus F protein were immunogenic in mice and cotton rats when delivered intranasally. The extent of responses were comparable to those elicited from wild type hMPV infection. Robust protein expressions by VRP were confirmed by immunostaining of infected BHK cells with polyclonal hMPV antisera. When these VRPs were inoculated into mice and cotton rat intranasally, they elicited significant amount of hMPV-specific IgA antibodies in both the upper and lower respiratory tracts. Local IgA secretion on the mucosal surfaces was traditionally shown to protect individuals from respiratory infections. Moreover, systemic IgG antibodies against F or G antibodies were detected in vaccinated animals. These antibodies also possessed neutralizing activity against hMPV. The cross-neutralizing activities of sera from VRP-vaccinated animals between different strains of the viruses were variable. Since the hMPV F sequences were constructed from sequence obtained from hMPV A2 clinical isolates, neutralizing activity towards the homologous A2 strain was the highest. There was a significant, but lower, neutralizing antibody titer towards hMPV A1 strain. Surprisingly, serum from VRP vaccinated animals did not neutralize hMPV subgroup B viruses at dilution as low as 1:20, given that the homology of the F gene between the subgroups are >95%. The difference in hMPV F sequences between the subgroups, although small, may contribute to conformational structure differences that is important for neutralization and renders further investigation.

More surprising is that the presence of higher titers of hMPV G-specific antibodies in vaccinated animals did not neutralize hMPV. Unlike RSV, the G protein did not seem to be a neutralizing antigen for hMPV and did not contribute to protection against challenge. The lack of neutralizing antibodies induction was demonstrated recently by the inventors using purified hMPV G protein as immunogen in cotton rats (unpublished data) and by another group using PIV to deliver hMPV G protein in hamsters (Skiadopoulos et al., 2006). The role of hMPV G protein in viral pathogenesis is still not defined, although the speculation of attachment and immuno-modulation properties similar to that of RSV G protein was proposed (Tripp et al., 2001; Bukreyev et al., 2006; Polack et al., 2005).

When mice or cotton rats vaccinated with VRP encoding hMPV F gene were challenged with wild-type hMPV, the challenge virus replication was reduced to lower than detectable levels in the lungs. The reduction correlated well with the level of hMPV serum neutralizing titer in the animals. This is synonymous with what was seen in RSV, in which a RSV serum neutralizing titer >380 was able to protect animals and humans from RSV challenge or infection (Prince et al., 1985). The challenge hMPV titer in the nose, however, was not completely reduced to undetectable levels in some animals. VRP-MPV.F vaccinated animals did have a significantly reduced titers in the nasal turbinates, possibly due to the presence of mucosal IgA antibodies. The incomplete protection of the nose could be due to several factors. One is that hMPV-specific IgA level in the nose was induced at a lower level than in the lungs. In the lungs, both hMPV-specific IgA in the BAL fluids and serum Ig antibodies contribute to protection while in the nose, hMPV-specific IgA was solely responsible for protection. Second, cellular immune responses may be important in reducing viral replication in the nasal turbinate. In RSV animal model, both RSV-specific CD4+ and CD8+ cells were found to be important in conferring protection in naïve animals against RSV challenge via adoptive transfer experiments (Cannon et al., 1988; Plotnicky-Gilquin et al., 2002). Therefore, cellular immunity may also contribute partly to protection in the upper respiratory tract. However, in our experience, cellular immunity was not found against the hMPV F protein in DBA/2 animals (data not shown). Several groups have also found limited cytotoxic T-cell response against hMPV F protein. T-cell epitopes were found restricted exclusively to M2-1 protein (Melendi et al., 2007) and M2-2 protein in H-2^{d} MHC-I alleles and N protein in H-2^{b} MHC-I alleles (Herd et al., 2006). It is, however, possible that cellular response against hMPV F would be found in the diverse MHC alleles in humans.

One concern for paramyxovirus vaccines is that they would enhance pulmonary disease and induce biased Th2 responses when immunized individual is exposed to natural infection. This is the case for formalin-inactivated RSV vaccine in infants and more recently formalin-inactivated hMPV vaccine in cotton rats (Yim et al., 2007). The inventors therefore evaluated lung histopathology and cytokine gene expression in VRP-vaccinated animals after wild type hMPV challenge. In this study, mice vaccinated with VRP had reduced inflammation and mucus production compared to unvaccinated animals. Vaccinated animals had minimal alveolar, peribronchiolar and perivascular infiltrates and no significant airway mucus production. Unvaccinated animals did show minor increases in lung inflammation with mild lymphocytic infiltration with a histopathology score slightly higher than that of the VRP-MPV.F immunized groups. Cytokine gene expressions were increased among all hMPV-infected animals compared to uninfected controls. However, the increase in IFN-γ gene expression was lower when comparing animal vaccinated with VRP-MPV.F to other groups. This may be due to the absence of T cells towards hMPV F protein. In the case of RSV, pulmonary disease is aggravated by T-cell responses in animal models (Cannon et al., 1988; Varga et al., 2001). This finding suggests that humoral response against hMPV did not predispose animals to imbalance immune responses in vaccinated animals against hMPV exposure.

### II. Paramyxoviruses

Paramyxoviruses are viruses of the Paramyxoviridae family of the Mononegavirales order; they are negative-sense single-stranded RNA viruses responsible for a number of human and animal diseases. Virions are enveloped and can be spherical, filamentous or pleomorphic. Fusion proteins and attachment proteins appear as spikes on the virion surface. Matrix proteins inside the envelope stabilise virus structure. The nucleocapsid core is composed of the genomic RNA, nucleocapsid proteins, phosphoproteins and polymerase proteins.

The genome consists of a single segment of negative-sense RNA, 15-19 kilobases in length and containing 6-10 genes. Extracistronic (non-coding) regions include: a 3′ leader sequence, 50 nucleotides in length which acts as a transcriptional promoter; and a 5′ trailer sequence, 50-161 nucleotides long. Intergenomic regions between each gene which are three nucleotides long for morbillivirus, respirovirus and henipavirus, variable length (1-56 nucleotides) for rubulavirus and pneumovirinae. Each gene contains transcription start/stop signals at the beginning and end which are transcribed as part of the gene. Gene sequences within the genome are conserved across the family due to a phenomenon known as transcriptional polarity (see Mononegavirales) in which genes closest to the 3′ end of the genome are transcribed in greater abundance than those towards the 5′ end. This mechanism acts as a form of transcriptional regulation. The gene sequence is: Nucleocapsid-Phosphoprotein-Matrix-Fusion-Attachment-Large (polymerase).

The nucleocapsid protein associates with genomic RNA (one molecule per hexamer) and protects the RNA from nuclease digestion. The phosphoprotein binds to the N and L proteins and forms part of the RNA polymerase complex. The matrix protein assembles between the envelope and the nucleocapsid core, it organises and maintains virion structure. The fusion protein projects from the envelope surface as a trimer, and mediates cell entry by inducing fusion between the viral envelope and the cell membrane by class I fusion. One of the defining characteristics of members of the paramyxoviridae family is the requirement for a neutral pH for fusogenic activity. The cell attachment proteins (H/HN/G) span the viral envelope and project from the surface as spikes. Many have been shown to bind to sialic acid on the cell surface and facilitate cell entry. Proteins are designated H for morbilliviruses and henipaviruses as they possess haemagglutination activity, observed as an ability to cause red blood cells to clump. HN attachment proteins occur in respiroviruses and rubulaviruses. These possess both haemagglutination and neuraminidase activity which cleaves sialic acid on the cell surface, preventing viral particles from reattaching to previously infected cells. Attachment proteins with neither haemagglutination nor neuraminidase activity are designated G (glycoprotein). These occur in members of pneumovirinae. The large protein is the catalytic subunit of RNA dependent RNA polymerase (RDRP).

The subfamily Pneumovirinae contains two important human pathogens, respiratory syncytial virus from the genus Pneumovirus, and metapneumovirus from the genus Metapneumovirus. Virions have an envelope and a nucleocapsid and are spherical to pleomorphic; however, filamentous and other forms are common. The virions are about 60-300 nm in diameter and 1000-10000 nm in length. The Mr of the genome constitutes 0.5% of the virion by weight. The genome is not segmented and contains a single molecule of linear negative-sense, single-stranded RNA. Virions may also contain occasionally a positive sense single-stranded copy of the genome. The complete genome is about 15,300 nucleotides long.

A. RSV

Human respiratory syncytial virus (hRSV) is a negative-sense, single-stranded RNA virus that causes respiratory tract infections in patients of all ages. It is the major cause of lower respiratory tract infection during infancy and childhood. In temperate climates there is an annual epidemic during the winter months. In tropical climates, infection is most common during the rainy season. In the United States, 60% of infants are infected during their first RSV season, and nearly all children will have been infected with the virus by 2-3 years of age. Natural infection with RSV does not induce protective immunity, and thus people can be infected multiple times. Sometimes an infant can become symptomatically infected more than once even within a single RSV season. More recently, severe RSV infections have increasingly been found among elderly patients as well.

For most people, RSV produces only mild symptoms, often indistinguishable from common colds and minor illnesses. The Centers for Disease Control consider RSV to be the “most common cause of bronchiolitis and pneumonia among infants and children under 1 year of age.” For some children, RSV can cause bronchiolitis, leading to severe respiratory illness requiring hospitalization and, rarely, causing death. This is more likely to occur in patients that are immunocompromised or infants born prematurely. Other RSV symptoms common among infants include listlessness, poor or diminished appetite, and a possible fever.

Recurrent wheezing and asthma are more common among individuals who suffered severe RSV infection during the first few months of life than among controls; whether RSV infection sets up a process that leads to recurrent wheezing or whether those already predisposed to asthma are more likely to become severely ill with RSV is a matter of considerable debate.

As the virus is ubiquitous in all parts of the world, avoidance of infection is not possible. Epidemiologically, a vaccine would be the best answer. Unfortunately, vaccine development has been fraught with spectacular failure and with difficult obstacles. Researchers are working on a live, attenuated vaccine, but at present no vaccine exists. However, palivizumab (brand name Synagis), a moderately effective prophylactic drug is available for infants at high risk. Palivizumab is a monoclonal antibody directed against RSV proteins. It is given by monthly injections, which are begun just prior to the RSV season and are usually continued for five months. RSV prophylaxis is indicated for infants that are premature or have either cardiac or lung disease.

Ribavirin, a broad-spectrum antiviral agent, was once employed as adjunctive therapy for the sickest patients; however, its efficacy has been called into question by multiple studies, and most institutions no longer use it. Treatment is otherwise supportive care only with fluids and oxygen until the illness runs its course. Amino acid sequences 200-225 and 255-278 of the F protein of human respiratory syncytial virus (HRSV) are T cell epitopes (Corvaisier et al., 1993). Peptides corresponding to these two regions were synthesized and coupled with keyhole limpet haemocyanin (KLH). The two conjugated proteins were administered intranasally to BALB/c mice alone or together with cholera toxin B (CTB). ELISAs revealed that the mixture of the conjugates with CTB increased not only the systemic response but also the mucosal immune response of the saliva. The systemic response was lower and the mucosal immune response was undetectable in mice immunized with the conjugates on their own. These results suggest that these two peptide sequences are effective epitopes for inducing systemic and mucosal immune responses in conjunction with CTB, and may provide the basis for a nasal peptide vaccine against RSV for human use.

B. MPV

Human metapneumovirus (hMPV) was isolated for the first time in 2001 in the Netherlands by using the RAP-PCR technique for identification of unknown viruses growing in cultured cells. hMPV is a negative single-stranded RNA virus of the family Paramyxoviridae and is closely related to the avian metapneumovirus (AMPV) subgroup C. It may be the second most common cause (after the RSV) of lower respiratory infection in young children.

Compared with RSV, infection with human metapneumovirus tends to occur in slightly older children and to produce disease that is less severe. Co-infection with both viruses can occur, and is generally associated with worse disease. Human metapneumovirus accounts for approximately 10% of respiratory tract infections that are not related to previously known etiologic agents. The virus seems to be distributed worldwide and to have a seasonal distribution with its incidence comparable to that for the influenza viruses during winter. Serologic studies have shown that by the age of five, virtually all children have been exposed to the virus and reinfections appear to be common. Human metapneumovirus may cause mild respiratory tract infection however small children, elderly and immunocompromised individuals are at risk of severe disease and hospitalization. The genomic organisation of hMPV is analogous to RSV, however hMPV lacks the non-structural genes NS1 and NS2 and the hMPV antisense RNA genome contains eight open reading frames in slightly different gene order than RSV (viz. 3′-N-P-M-F-M2-SH-G-L-5′). hMPV is genetically similar to the avian pneumoviruses A, B and in particular type C. Phylogenetic analysis of HMPV has demonstrated the existence of two main genetic lineages termed subtype A and B containing within them the subgroups A1/A2 and B1/B2 respectively. The identification of HMPV has predominantly relied on reverse-transcriptase polymerase chain reaction (RT-PCR) technology to amplify directly from RNA extracted from respiratory specimens. Alternative more cost effective approaches to the detection of hMPV by nucleic acid-based approaches have been employed and these include: 1) detection of hMPV antigens in nasopharyngeal secretions by immunofluorescent-antibody test 2) the use of immunofluorescence staining with monoclonal antibodies to detect hMPV in nasopharyngeal secretions and shell vial cultures 3) immunofluorescence assays for detection of hMPV-specific antibodies 4) the use of polycloncal antibodies and direct isolation in cultures cells.

### III. VEE Vaccine Delivery System

The present invention utilizes, in one aspect, an alphavirus delivery system based on virus replicon particles (VRPs) of venezuelan equine encephalitis (VEE) virus, an RNA virus of the Togaviradae family VRPs are non-replicating particles developed by Pushko et al. in 1997, which been used successfully and safely in immunization and challenge studies for a wide range of viral and bacterial pathogens in animal model systems (Pushko et al., 1997; Balasuriya et al., 2002; Burkhard et al., 2002; Gipson et al., 2003; Harrington et al., 2002; Hevey et al., 1998; Johnston et al., 2005; Lee et al., 2002; Pushko et al., 2001; Schultz-Cherry et al., 2000; Velders et al., 2001; Wang et al., 2005), including influenza virus, Lassa fever virus, Marburg virus, and most recently HIV. Importantly, these particles have been shown to induce mucosal immune responses after parenteral or intradermal inoculation in animals (Harrington et al., 2002; Davis et al., 1996). Currently this vector system is being tested in phase I clinical trials in humans to determine the safety of candidate vaccine encoding HIV antigens (Davis et al., 2002; Williamson et al., 2003).

VRPs are intact, replication-deficient VEE virus particles that contain a modified positive-sense RNA viral genome designed to express only the heterologous antigens. These particles are produced in a cellular packaging system in which structural proteins are supplied in trans and only the modified viral genome is packaged into an intact VRP. The resulting replicons express high levels of antigens in infected cells and induce humoral and cellular immune responses in vivo (Pushko et al., 1997). VRPs possess the ability to target dendritic cells and induce mucosal responses (MacDonald and Johnston, 2000), which is optimal for protecting against viruses at the respiratory tract mucosa. Although the mechanism underlying this unique mucosal immunogenicity of VRPs is not completely understood, significant numbers of cells secreting antigen-specific IgA have been detected in the mucosa in immunized animals following VRP immunization (Pushko et al., 1997; Harrington et al., 2002; Johnston et al., 2005; Davis et al., 1996; Davis et al., 2002). Moreover, when VRP particles were co-administered with microbial antigens, they exhibit adjuvant activity in the systemic and mucosal immune compartments (Thompson et al., 2006).

The present inventors have generated VEE replicon vaccine vectors for both RSV and hMPV and tested them to determine whether effective mucosal protection could be induced against these pathogens following intranasal immunization. VRPs encoding the RSV F protein induced both systemic and mucosal antibody responses. These VRPs also induced antigen-specific T cells in both the lungs and spleens of immunized animals. The T cell responses were Th1/Th2 balanced, and aggravated histopathology was not observed. In addition, these animals were protected completely following challenge with wild-type RSV. In contrast, animals vaccinated with VRPs encoding the RSV attachment protein G were only partially protected. These findings provide proof-of-principle that VEE VRPs expressing the RSV F protein can be used to prevent RSV infection.

Additional details of this vector system and its use can be found in U.S. Patent Publication 2002/014975 A1 (incorporated by reference), as well as on the World Wide Web at alphavax.com. Other patent documents that are relied upon to provide a description of this system include U.S. Pat. Nos. 5,185,440, 5,505,947, 5,643,576, 5,792,462, 6,156,558, 6,521,235, 6,531,135, 6,541,010, 6,738,939, 7,045,335 and 7,078,218, each of which are incorporated herein by reference.

The following discussion is derived from U.S. Pat. No. 7,045,335:
The terms “alphavirus replicon particles,” “virus replicon particles” or “recombinant alphavirus particles,” used interchangeably herein, mean a virion-like structural complex incorporating an alphavirus replicon RNA that expresses one or more heterologous RNA sequences. Typically, the virion-like structural complex includes one or more alphavirus structural proteins embedded in a lipid envelope enclosing a nucleocapsid that in turn encloses the RNA. The lipid envelope is typically derived from the plasma membrane of the cell in which the particles are produced. Preferably, the alphavirus replicon RNA is surrounded by a nucleocapsid structure comprised of the alphavirus capsid protein, and the alphavirus glycoproteins are embedded in the cell-derived lipid envelope. The alphavirus replicon particles are infectious but replication-defective, i.e., the replicon RNA cannot replicate in the host cell in the absence of the helper nucleic acid(s) encoding the alphavirus structural proteins.
As described in detail hereinbelow, the present invention provides improved alphavirus-based replicon systems that reduce the potential for replication-competent virus formation and that are suitable and/or advantageous for commercial-scale manufacture of vaccines or therapeutics comprising them. The present invention provides improved alphavirus RNA replicons and improved helpers for expressing alphavirus structural proteins.
In one embodiment of this invention, a series of “helper constructs,” i.e., recombinant DNA molecules that express the alphavirus structural proteins, is disclosed in which a single helper is constructed that will resolve itself into two separate molecules in vivo. Thus, the advantage of using a single helper in terms of ease of manufacturing and efficiency of production is preserved, while the advantages of a bipartite helper system are captured in the absence of employing a bipartite expression system. In one set of these embodiments, a DNA helper construct is used, while in a second set an RNA helper vector is used. In the case of the DNA helper constructs that do not employ alphaviral recognition signals for replication and transcription, the theoretical frequency of recombination is lower than the bipartite RNA helper systems that employ such signals.
In the preferred embodiments for the constructs of this invention, a promoter for directing transcription of RNA from DNA, i.e., a DNA dependent RNA polymerase, is employed. In the RNA helper embodiments, the promoter is utilized to synthesize RNA in an in vitro transcription reaction, and specific promoters suitable for this use include the SP6, T7, and T3 RNA polymerase promoters. In the DNA helper embodiments, the promoter functions within a cell to direct transcription of RNA. Potential promoters for in vivo transcription of the construct include eukaryotic promoters such as RNA polymerase II promoters, RNA polymerase III promoters, or viral promoters such as MMTV and MoSV LTR, SV40 early region, RSV or CMV. Many other suitable mammalian and viral promoters for the present invention are available in the art. Alternatively, DNA dependent RNA polymerase promoters from bacteria or bacteriophage, e.g., SP6, T7, and T3, may be employed for use in vivo, with the matching RNA polymerase being provided to the cell, either via a separate plasmid, RNA vector, or viral vector. In a specific embodiment, the matching RNA polymerase can be stably transformed into a helper cell line under the control of an inducible promoter. Constructs that function within a cell can function as autonomous plasmids transfected into the cell or they can be stably transformed into the genome. In a stably transformed cell line, the promoter may be an inducible promoter, so that the cell will only produce the RNA polymerase encoded by the stably transformed construct when the cell is exposed to the appropriate stimulus (inducer). The helper constructs are introduced into the stably transformed cell concomitantly with, prior to, or after exposure to the inducer, thereby effecting expression of the alphavirus structural proteins. Alternatively, constructs designed to function within a cell can be introduced into the cell via a viral vector, e.g., adenovirus, poxvirus, adeno-associated virus, SV40, retrovirus, nodavirus, picornavirus, vesicular stomatitis virus, and baculoviruses with mammalian pol II promoters.
Once an RNA transcript (mRNA) encoding the helper or RNA replicon vectors of this invention is present in the helper cell (either via in vitro or in vivo approaches, as described above), it is translated to produce the encoded polypeptides or proteins. The initiation of translation from an mRNA involves a series of tightly regulated events that allow the recruitment of ribosomal subunits to the mRNA. Two distinct mechanisms have evolved in eukaryotic cells to initiate translation. In one of them, the methyl-7-G(5′)pppN structure present at the 5′ end of the mRNA, known as “cap,” is recognized by the initiation factor eIF4F, which is composed of eIF4E, eIF4G and eIF4A. Additionally, pre-initiation complex formation requires, among others, the concerted action of initiation factor eIF2, responsible for binding to the initiator tRNA-Met₁, and eIF3, which interacts with the 40S ribosomal subunit (reviewed in Hershey & Merrick, 2000.)
In the alternative mechanism, translation initiation occurs internally on the transcript and is mediated by a cis-acting element, known as an internal ribosome entry site (IRES), that recruits the translational machinery to an internal initiation codon in the mRNA with the help of trans-acting factors (reviewed in Jackson, 2000). During many viral infections, as well as in other cellular stress conditions, changes in the phosphorylation state of eIF2, which lower the levels of the ternary complex eIF2-GTP-tRNA-Met.sub.1, results in overall inhibition of protein synthesis. Conversely, specific shut-off of cap-dependent initiation depends upon modification of eIF4F functionality (Thompson & Sarnow, 2000).
IRES elements bypass cap-dependent translation inhibition; thus the translation directed by an IRES is termed “cap-independent.” Hence, IRES-driven translation initiation prevails during many viral infections, for example picornaviral infection (Macejak & Sarnow, 1991). Under these circumstances, cap-dependent initiation is inhibited or severely compromised due to the presence of small amounts of functional eIF4F. This is caused by cleavage or loss of solubility of eIF4G (Gradi et al., 1998); 4E-BP dephosphorylation (Gingras et al., 1996) or poly(A)-binding protein (PABP) cleavage (Joachims et al., 1999).
IRES sequences have been found in numerous transcripts from viruses that infect vertebrate and invertebrate cells as well as in transcripts from vertebrate and invertebrate genes. Examples of IRES elements suitable for use in this invention include: viral IRES elements from Picornaviruses, e.g., poliovirus (PV), encephalomyocarditis virus (EMCV), foot-and-mouth disease virus (FMDV), from Flaviviruses, e.g., hepatitis C virus (HCV), from Pestiviruses, e.g., classical swine fever virus (CSFV), from Retroviruses, e.g., murine leukemia virus (MLV), from Lentiviruses, e.g., simian immunodeficiency virus (SIV), or cellular mRNA IRES elements such as those from translation initiation factors, e.g., eIF4G or DAP5, from Transcription factors, e.g., c-Myc (Yang and Sarnow, 1997) or NF-κB-repressing factor (NRF), from growth factors, e.g., vascular endothelial growth factor (VEGF), fibroblast growth factor (FGF-2), platelet-derived growth factor B (PDGF B), from homeotic genes, e.g., Antennapedia, from survival proteins, e.g., X-Linked inhibitor of apoptosis (XIAP) or Apaf-1, or chaperones, e.g., the immunoglobulin heavy-chain binding protein BiP (reviewed in Martinez-Salas et al., 2001.)
Preferred IRES sequences that can be utilized in these embodiments are derived from: encephalomyocarditis virus (EMCV, accession # NC001479), cricket paralysis virus (accession # AF218039), Drosophila C virus accession # AF014388, Plautia stali intestine virus (accession # AB006531), Rhopalosiphum padi virus (accession # AF022937), Himetobi P virus (accession # AB017037), acute bee paralysis virus (accession # AF150629), Black queen cell virus (accession # AF183905), Triatoma virus (accession # AF178440), Acyrthosiphon pisu virus (accession # AF024514), infectious flacherie virus (accession # AB000906), and Sacbrood virus (accession # AF092924). In addition to the naturally occurring IRES elements listed above, synthetic IRES sequences, designed to mimic the function of naturally occurring IRES sequences, can also be used. In the embodiments in which an IRES is used for translation of the promoter driven constructs, the IRES may be an insect TRES or another non-mammalian IRES that is expressed in the cell line chosen for packaging of the recombinant alphavirus particles, but would not be expressed, or would be only weakly expressed, in the target host. In those embodiments comprising two IRES elements, the two elements may be the same or different.

The entire passage above is specifically incorporated herein by reference.

### IV. Proteins for Use in VEE Vectors

Various RSV and hMPV proteins can be utilized in the VEE vaccine delivery system discussed above. In particular, the F and G proteins of both RSV and the F protein of MPV are contemplated as appropriate antigens. The sequences for these four proteins are appended hereto as SEQ ID NOS: 2, 4, and 6.

In addition to the use of full length sequences, the present invention contemplates the use of various nucleic acid that encode fragments and truncated versions of these proteins, including a soluble version that lacks the transmembrane domain of the native protein. For example, nucleic acid encoding a portion of the protein as set forth in SEQ ID NO: 2, SEQ ID NO: 4, or SEQ ID NO: 6 may be used in various embodiments of the invention. In certain embodiments, a fragment of the may comprise, but is not limited to about 50, about 75, about 100, about 110, about 120, about 130, about 140, about 150, about 160, about 170, about 180, about 190, about 200, about 210, about 220, about 230, about 240, about 250 or more residues, and any range derivable therein.

It also will be understood that such partial sequences, along with full length sequences, may be joined or fused to additional coding regions, such as those for additional N- or C-terminal amino acids, and yet still be essentially as set forth in one of the sequences disclosed herein. One example is fusion to a carrier protein that can improve immunogenicity of the viral sequences.

### IV. Formulations and Administration

The phrases “pharmaceutically acceptable” or “pharmacologically acceptable” refer to molecular entities and compositions that do not produce an adverse, allergic, or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents, and the like. The use of such reagents for pharmaceutical substances is well known in the art. Except insofar as any conventional agent is incompatible with the active ingredients, its use in the therapeutic compositions is contemplated. Supplementary active ingredients, such as adjuvants or biological response modifiers, can also be incorporated into the administration.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and regimen. The quantity to be administered, both according to number of treatments and unit dose, depends on the protection desired.

For viral vectors, particularly attenuated viral vectors, one generally will prepare a viral vector stock of high titer. Depending on the titer attainable, one will deliver 1 to 100, 10 to 50, 100-1000, or up to 1×10⁴, 1×10⁵, 1×10⁶, 1×10⁷, 1×10⁸, 1×10⁹, 1×10¹⁰, 1×10¹¹, 1×10¹², 1×10¹³ or 1×10¹⁴ infectious particles to the patient. Formulation as a pharmaceutically acceptable composition is discussed below above.

B. Vaccination Protocols

The vaccines of the present invention can be formulated for parenteral administration, e.g., formulated for injection via the intradermal, intravenous, intramuscular, subcutaneous, or even intraperitoneal routes. Administration by the intradermal and intramuscular routes are specifically contemplated. The vaccine could alternatively be administered by a topical route directly to the mucosa, for example by nasal drops, inhalation, or by nebulizer. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intradermal, and intraperitoneal administration. In this connection, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, Remington's Pharmaceutical Sciences, 1990). Some variation in dosage will necessarily occur depending on the age and possibly medical condition of the subject being treated. The person responsible for administration will, in any event, determine the appropriate dose for the individual subject.

In many instances, it will be desirable to have several or multiple administrations of the vaccine. The compositions of the invention may be administered 1, 2, 3, 4, 5, 6, 7, 8, 9, 10, or more times. The administrations will normally be at from one to twelve week intervals, more usually from one to four week intervals. Periodic re-administration will be desirable with recurrent exposure to the pathogen.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Animals and Cell Lines. Specific pathogen-free 5-6 week old BALB/c mice and cotton rats were purchased from Harlan (Indianapolis, Ind.). Animals were housed in micro-isolator cages throughout the study. All experimental procedures performed were approved by the Institutional Use and Care of Animals Committee at Vanderbilt University Medical Center.

HEp-2 cells were obtained from ATCC (CCL-23) and maintained in OptiMEM medium (Invitrogen, CA) supplemented with 2% fetal bovine serum (FBS), 4 mM L-glutamine, 5 μg/mL amphotericin B and 50 μg/mL gentamicin sulfate at 37° C. with 5% CO₂.

VEE Constructs and Generation of VRPs encoding RSV F or G genes. The method of construction and packaging of VRPs was described (Davis et al., 1996). A VEE-based replicon, pVR21, which was derived from mutagenesis of a cDNA clone of the Trinidad donkey stain of VEE was used to insert heterologous genes. RSV F, G or human metapneumovirus (hMPV) F genes optimized for mammalian cell expression were cloned into pVR21 downstream of the subgenomic 26S promoter via a two-step PCR and ligation process. First, pVR21 DNA was PCR-amplified with primers to generate amplicons that included a unique 5′ SwaI restriction site and the 26S mRNA leader at the 3′ end of the amplicon. Second, the RSV F, G or hMPV F gene was PCR-amplified to obtain amplicons that contained the 26S mRNA leader at the 5′ end, the heterologous gene, and a PacI restriction site at the 3′ end. The two amplicons then were used as template for a third PCR using a forward primer hybridizing to the pVR21 amplicon and a reverse primer hybridizing to the RSV F, G or hMPV F amplicon. This PCR generated an overlapping fragment that spanned the 26S promoter leader sequence, the RSV F, G or hMPV F sequences and contained the unique 5′ SwaI and 3′ PacI restriction sites that could be directionally ligated back into a digested pVR21 plasmid.

For generation of VRPs, capped RNA transcripts of pVR21 containing RSV F, G or hMPV F genes were generated in vitro with the mMESSAGE mMACHINE T7 kit (Ambion, Austin, Tex.). Similarly, helper transcripts that encoded the VEE capsid and glycoproteins genes were generated in vitro. Baby hamster kidney (BHK) cells then were co-transfected by electroporation with the pVR21 and helper RNAs and culture supernatants were harvested at 30 hours after transfection.

VRP Titration. Serial dilutions of VRPs encoding RSV F (designated VRP-RSV.F) or RSV G (designated VRP-RSV.G) were used to infect BHK cells in eight-chamber slides (Nunc) for 20 hours at 37° C. Infected BHK cells were fixed and immunostained for VEE proteins. Infectious units then were calculated from the number of VEE glycoprotein-stained cells per dilution and converted to infectious units (IU) per milliliter.

Western Blot. BHK cells were infected at a moi of 5 with VRP-RSV.F, VRP-RSV.G or VRP-MPV.F for 24 hours at 37° C. Infected BHK cells were washed twice with ice-cold PBS and scraped into microfuge tubes. The cells were pelleted for 10 seconds at 6000 rpm and lysed in lysis buffer (50 mM Tris-HCl, 150 mM NaCl, 1% Triton X-100, 0.5% v/v protease inhibitor cocktail, pH 8.0) (Sigma, St. Louis, Mo.) for 10 minutes on ice. The resulting cell lysates then were cleared from debris by centrifugation at 13,000 rpm for 5 minutes.

Proteins were separated by electrophoresis using a NuPAGE 4-12% Bis-Tris gel (Novex) and transferred onto an Invitrolon PVDF membrane (Invitrogen). The membrane was blocked with TBST/5% non-fat dry milk at 4° C. overnight. The blot then was washed and stained for the presence of RSV F or RSV G proteins with mouse monoclonal antibodies (1:1000 dilution in TBST/1% non-fat dry milk) for an hour at room temperature. After the primary antibody incubation, secondary goat anti-mouse HRP-conjugated antibodies (1:5000 dilution in TBST/1% non-fat dry milk) were added. The blot was washed again with TBST after a one-hour incubation and developed using SuperSignal West Pico chemiluminescent substrate (Pierce, Rockford, Ill.).

Immunofluorescence staining. BHK cells were infected at a moi of 5 with VRP-RSV.F or VRP-RSV.G in eight-chamber slides (Nunc) for 24 hours at 37° C. Infected BHK cells were fixed in 80% methanol for an hour at 4° C. The cells then were blocked with PBS/3% BSA for two hours at room temperature. Primary antibodies against RSV F or RSV G (1:1000 dilution in PBS/1% BSA) were added and allowed to incubate for an hour at room temperature. Cells were washed extensively after the primary antibodies incubation with TBST and secondary goat anti-mouse AlexaFluor C555-conjugated antibodies were added (1:1000 dilution in TBST/1% BSA) to the cells for an additional hour. The slide then was washed with TBST and mounted with Prolong antifade medium (Invitrogen). The slide was visualized under a LSM510 inverted laser scanning confocal microscope (Carl Zeiss Microimaging, Thornwood, N.Y.).

Vaccination and Challenge of Mice or Cotton Rats. BALB/c mice were anesthetized with isoflurane by inhalation and vaccinated intranasally with various titers of VRP-RSV.F or VRP-RSV.G in a 100 μl inoculum. Control groups were inoculated with phosphate buffered saline (PBS), 5×10⁵ PFU of RSV wild-type strain A2 or 10⁶ infectious units of VRP-MPV.F via the same route. Mice that were vaccinated with VRPs were boosted with the same dose two and four weeks later. The mice were observed for clinical signs daily and bled at 14 day intervals to follow immune responses.

Twenty eight days after the third immunization, mice from all groups were challenged with 5×10⁵ PFU of RSV wild-type strain A2 intranasally. To monitor virus replication in the upper and lower respiratory tracts, nasal turbinates and lungs were harvested on day 4 post challenge and subsequently assayed for virus titer. Similarly, cotton rats were vaccinated on day 0 and day 14 with 10⁶ IU of VRP-RSV.F or VRP-RSV.G intranasally in groups of 4. Control groups were vaccinated with PBS, 5×10⁵ PFU of RSV A2 or 10⁶ IU of VRP-MPV.F. They then were bled on day 35 to monitor immune responses and were challenged with 5×10⁵ PFU of RSV A2 on day 42 and sacrificed on day 46. Lung and nasal turbinates were harvested separately and homogenized to determine viral titers.

BAL Fluid and Nasal Wash Collection. A subset of animals was sacrificed on day 56 to collect bronchoalveolar lavage (BAL) fluids and nasal washes. BAL fluids were collected by ligation of the trachea with suture, insertion of a 23-gauge blunt needle into the distal trachea, followed by three in-and-out flushes of the airway with 1 mL of sterile PBS. Nasal washes were obtained by flushing 3 ml PBS through the upper trachea and out the nasal orifice into a sterile receptacle. Both BAL and nasal washes were concentrated 10-fold using 10 kD molecular weight cutoff Centricon concentrators (Millipore, Bedford, Mass.).

Splenocytes and Lung Lymphocytes Collection. Spleens were harvested from vaccinated and control mice 14 days after immunization. Spleens were placed in RPMI medium supplemented with 10% FBS, 10 mM HEPES buffer, 2 mM L-glutamine, 0.5 mg/ml gentamicin and 50 mM 2-mercaptoethanol (designated complete RPMI). The spleens were minced and grinded through cell strainers (Becton-Dickinson, San Jose, Calif.) to obtain single-cell suspensions. The cells then were lysed with red blood cell lysing buffer (Sigma-Aldrich, St Louis, Mo.) and washed with complete RPMI before use. Lungs were excised and washed in PBS once. The lungs were placed in complete RPMI, minced, grinded and passed through cell strainers. The resulting suspensions were underlaid with Ficoll gradient and centrifuged at 1000 rpm for 10 minutes. Buffy coats then were removed and lymphocytes were counted.

RSV F Protein-Specific ELISA. Sera collected at day 14, 28 or 42 were tested for the presence of F protein-specific antibodies. Concentrated nasal washes and BAL fluids also were tested. Briefly, 150 ng of purified recombinant RSV F protein was adsorbed onto Immulon 2B plates overnight in carbonate buffer (pH 9.8) at 4° C. The plate then was blocked with 1% bovine serum albumin (BSA) in PBS for 2 hours at room temperature. After thorough washing with TBST/1% BSA, serial dilutions of serum, nasal wash or BAL fluid samples were added to the plate and allowed to incubate for an hour at room temperature. The plates were washed again and horseperoxidase (HRP)-conjugated anti mouse IgA (1:500 dilution), IgG (1:5000 dilution), IgG1 (1:500 dilution) or IgG2a (1:500 dilution) antibodies were added (Southern Biotech, Birmingham, Ala.) and allowed to incubate for another hour. Finally, the plate was washed and 100 μl of One-Step Turbo TMB peroxidase substrate (Pierce, Rockford, Ill.) was added per well to quantify the relative amounts of F-specific IgA, IgG, IgG1 or IgG2a in the samples. The reactions then were stopped by adding 50 μl of 1M HCl and the absorbances of the samples were read at 450 nm.

Neutralizing Antibody Assay. Serum samples were tested for the presence of RSV neutralizing antibodies. Briefly, a viral suspension that was standardized to yield 50 plaques per well in HEp-2 cell monolayer cultures was used. An aliquot of the RSV suspension was incubated with serial dilutions of the serum samples. After an hour, the suspension was absorbed onto HEp-2 cells and then overlaid an hour later with a semisolid methylcellulose overlay. After 5 days, the cell culture monolayers were fixed and stained by immunoperoxidase using anti-F monoclonal antibodies to identify plaques. Plaques were counted and plaque reduction was calculated by regression analysis to provide a 60% plaque reduction titer.

Viral Plaque Titer Assay. Serial dilutions of nasal turbinates or lung homogenates were inoculated onto HEp-2 cell monolayer cultures and plaque assays were performed as described above.

Enzyme-linked immunosorbent spot (ELISPOT) assay. Interferon-γ secreting T cells were quantified in an ELISPOT assay. Briefly, 1 μg of anti-mouse IFN-γ capture antibody per well was adsorbed onto methanol-activated Millipore ELLIP 10SSP multiscreen plates overnight at 4° C. The plates then were washed three times with PBS and blocked with complete RPMI for 2 hours at room temperature. Peptides that correspond to a known MHC-restricted RSV F protein epitope, RSV G protein epitope or unrelated peptide epitope were added into each well in 50 μl volume. Freshly isolated splenocytes and lung lymphocytes then were added at a concentration of 2×10⁵ cells per well in 50 μl complete RPMI in duplicate. The plates were incubated for 20 hours at 37° C. in 5% CO₂ before harvest. On the day of harvest, the plates were washed three times with PBS-Tween and 0.2 μg of biotinylated anti-IFN-γ antibodies in PBS was added to each well, followed by a 3 hour incubation at room temperature. Plates were washed again before the addition of 100 μl of Avidin-Peroxidase Complex (Vector Laboratories, Burlingame, Calif.). Plates were washed after an hour at room temperature and 100 μl of AEC substrate was added to the plate. The substrate was allowed to incubate for 4 minutes at room temperature before the plates were rinsed in cold tap water. The plates then were air-dried overnight before spots were counted by an automatic reader (Cellular Technology, Cleveland, Ohio) and expressed as number of IFN-γ expressing cells per 10⁶ cells.

Histology. Four days after RSV challenge, mice were euthanized with CO₂ and lungs were harvested. To preserve structural integrity of the lungs, 1 ml of 10% neutral buffered formalin was instilled into the lungs via tracheotomy, followed by ligation of the trachea with suture. The whole lung then was immersed in 10% neutral buffered formalin overnight. After fixation, the lungs were dehydrated by immersing in 70% ethanol for another day. The lungs then were embedded in paraffin, sectioned and stained with hematoxylin/eosin or Periodic-Acid Schiff's solution. Mucus glycoconjugates were visualized by PAS staining. The severity of airway inflammation was graded group-blind on a 0-4 scale by a pathologist based on the following criteria: 0, no detectable airway inflammation; 1, less than 25% bronchials and surrounding vasculature were found to have either perivascular or peribronchial inflammatory cell infiltration; 2, approximately 25-50% of bronchials and surrounding vasculature were affected; 3, approximately 50-75% bronchials and surrounding vasculature were affected; 4, more than 75% of bronchials and surrounding vasculature were affected.

Cytokine gene expression in the lungs after RSV challenge. Lungs from unvaccinated or vaccinated mice were harvested 4 days after RSV challenge and placed into RNeasy RNA tissue lysis buffer (Qiagen). The tissues were homogenized and mRNAs were extracted according to manufacturer's protocol. Primers and probes were purchased from Applied Biosystems (Foster City, Calif.) to measure mRNA for Th1 or Th2 cytokines based on GenBank sequences for murine GAPDH, gamma interferon (IFN-γ) and interleukins 2 (IL-2), 4 (IL-4), 5 (IL-5), 10 (IL-10) and 12 (IL-12). Probes were labeled at the 5′ end with 6-carboxyfluorescein (FAM) and at the 3′ end with the nonfluorescent quencher Blackhole Quencher 1 (BHQ1; Operon Biotechnologies, Huntsville, Ala.). Reverse-transcribed real-time PCR was performed using Quantitect Probe RT-PCR kit (Qiagen, Valencia, Calif.) and a Smart Cycler II (Cepheid, Sunnyvale, Calif.) using 5 μl of extracted mRNA. The parameters used were 1 cycle of 50° C. for 2 min, 1 cycle of 95° C. for 10 min, and 40 cycles of 95° C. for 15 sec and 60° C. for 1 min. Reactions were performed in triplicate, with no template as negative control. Relative amounts of cytokine gene mRNAs were determined by normalizing to the level of GAPDH mRNA, and uninfected mice were used as baseline controls. Differences in mRNA levels were computed using the ΔΔCₜ method comparing infected to uninfected mice.

Statistics. GraphPad Prism software was used to analyze the data (GraphPad Software Inc., San Diego, Calif.). All data were expressed as the mean and standard error of the mean. Data also were analyzed by Mann-Whitney rank sum test to compare the sample means between any two experimental groups.

### Example 2

### Results

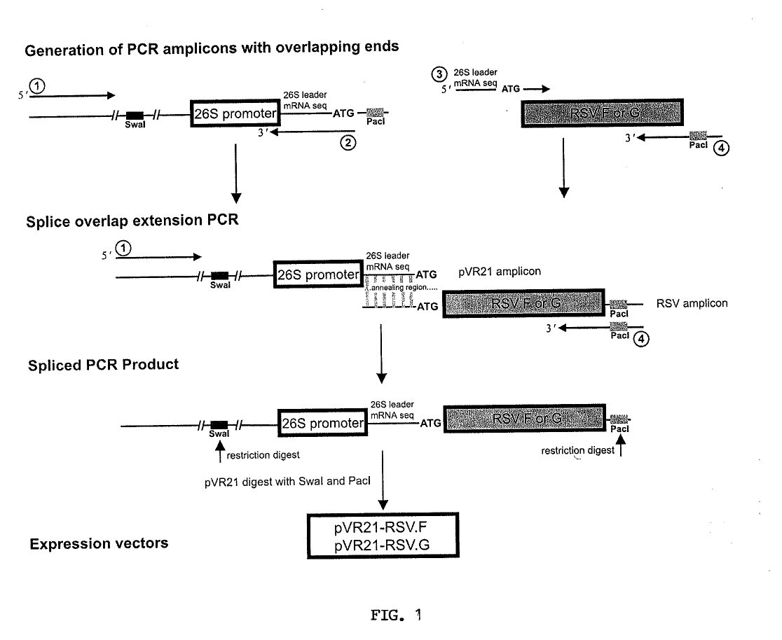

Cloning and expression of RSV antigens using VEE replicon particles (VRPs). RSV fusion (RSV.F) and attachment (RSV.G) glycoprotein genes were cloned into the pVR21 VEE replicon vector under the control of a subgenomic 26S promoter (FIG. 1). VRPs then were produced in BHK cells by cotransfecting the replicon vector with plasmids encoding VEE capsid and structural proteins.

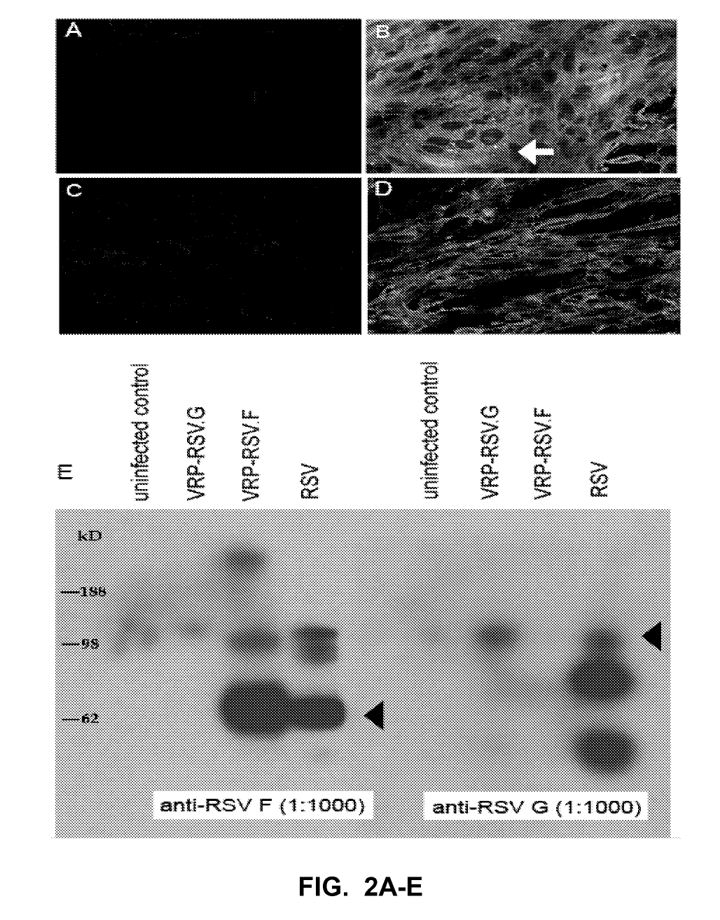

To ensure these replicons expressed the desired antigens, BHK cells were infected at a moi of 5 with VRPs. Antigen expression then was measured by Western blot and immunostaining with RSV.F or RSV.G specific monoclonal antibodies. A robust amount of RSV F protein was expressed, as evident by the intense staining of BHK cells with anti-RSV F antibodies (FIG. 2B), compared to uninfected control cells (FIG. 2A). Examination by confocal microscopy revealed the formation of syncytia when RSV F proteins were expressed (arrow, FIG. 2B). RSV F expression also was confirmed by Western blot of infected cell lysates, which showed a predicted band of RSV F at 60 kD (FIG. 2E).

Similarly, cells infected with VRP encoding RSV.G expressed the predicted antigens when immunostained with anti-RSV G antibodies (FIG. 2D) and on Western blot of cell lysates (FIG. 2E). Staining of cells infected with RSV.G VRP showed a membrane-bound pattern, which is consistent with previous reports of the distribution of G during RSV infection (Teng et al., 2001; Peroulis et al., 1999).

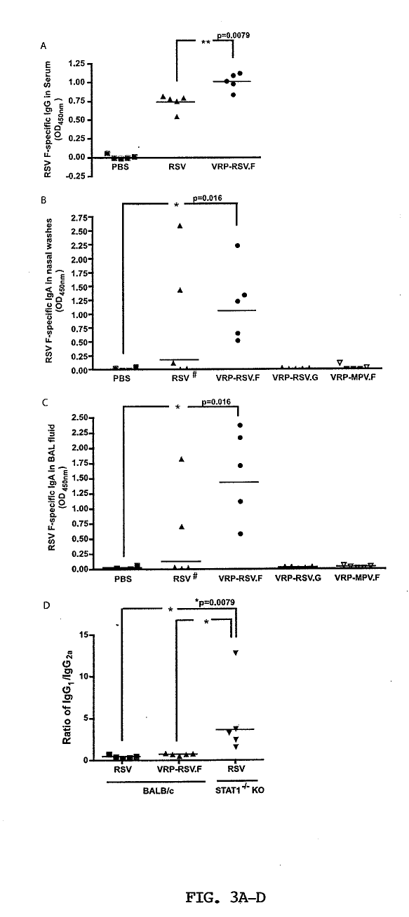

Systemic IgG and mucosal IgA responses in VRP-vaccinated mice. To assess if VRPs could induce systemic humoral immune responses, the inventors measured the titers of RSV F-specific IgG antibodies in the serum of vaccinated mice by ELISA. Intranasal inoculation of VRPs induced significantly higher titers of RSV F-specific IgG in the serum of vaccinated mice (1.4-fold higher) than in those infected once with RSV (FIG. 3A). Moreover, mucosal RSV F-specific IgA antibodies were detected in the nasal washes and bronchioalveolar lavage (BAL) fluids, which reflect the presence of mucosal immunity in the upper and lower respiratory tracts of vaccinated animals respectively (FIGS. 3B and 3C).

Isotype profile of the serum IgG response. Formalin-inactivated RSV and subunit protein vaccines induce aberrant immune responses in naïve subjects characterized by Th2-dominant cytokines and elevated IgG1 to IgG2a ratios. A Th2-dominant RSV response has also been noted in STAT-1-deficient mice (Durbin et al., 2002). The inventors tested whether animals vaccinated with VRPs will induce a balanced response as seen in those infected with wild-type RSV or an aberrant response as seen in RSV-infected STAT-1-deficient mice. RSV-infected and VRP-vaccinated BALB/c mice exhibited a serum IgG profile characteristic of a balanced Th1/Th2 response whereas STAT-1 knockout mice showed the predicted atypical Th2-biased response. The ratio of IgG1 to IgG2a was 4-fold lower for VRP-vaccinated and RSV-infected BALB/c mice compared to RSV-infected STAT-1 KO mice. A statistical significant difference between VRP-vaccinated group and RSV-infected BALB/c was not detected.

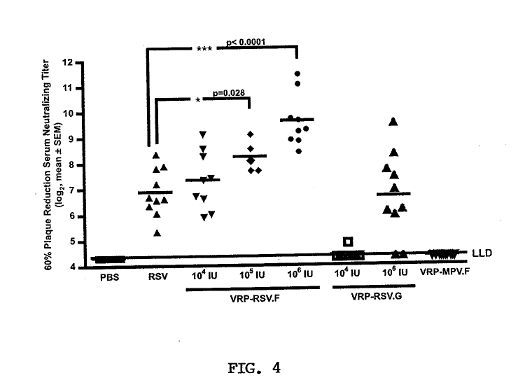

Serum RSV neutralizing activity in VRP-vaccinated animals. The presence of neutralizing antibodies in the serum is an important parameter that has been implicated to protect the lower respiratory tract against RSV infection (Murphy et al., 1988; Prince et al., 1985; Sami et al., 1995). The inventors therefore measured neutralizing activity of the sera from VRP-vaccinated mice and cotton rats using a 60% plaque reduction assay. Mice vaccinated with PBS or VRP expressing hMPV.F protein, which served as a heterologous virus control, did not induce any detectable neutralizing titer. Intranasal vaccination with VRP-RSV.F generated a 1.4- to 6.7-fold higher in serum neutralizing antibody titer compared to mice infected with RSV. The increases were dose-dependent and were significantly different in the 10⁵ and 10⁶ IU dose groups compared to the 10⁴ IU dose group. VRP-RSV.G vaccinated mice had a lower neutralizing titer than those vaccinated with VRP-RSV.F, which is consistent with previous observations of the relative immunogenicity of RSV F and G proteins. At high dose, the neutralizing activity was comparable to that of the sera of RSV-infected mice, but the low dose did not induce any detectable responses (FIG. 4A).

For cotton rats, intranasal vaccination with 10⁶ IU of VRP-RSV.F induced a serum neutralizing activity of 1:210 compared to 1:170 from RSV-infected animals (FIG. 4B).

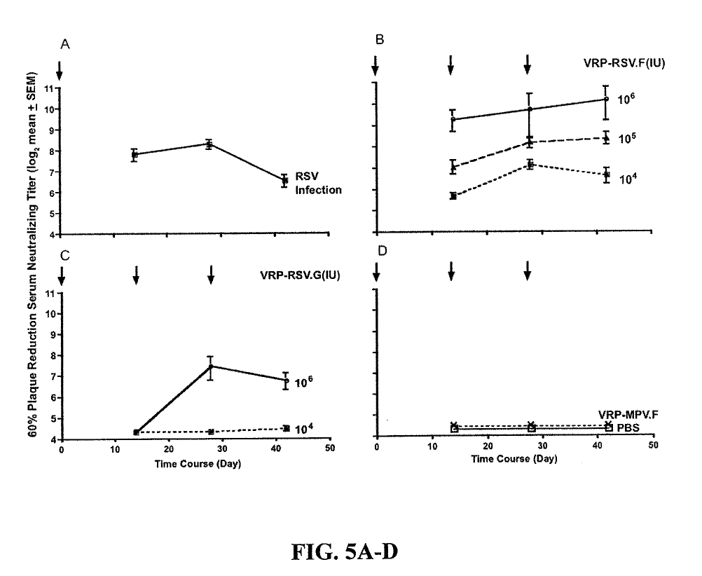

Kinetics of neutralizing activity after prime-boost immunization. The inventors measured serum neutralizing titers 2 weeks after each prime-boost vaccination. As predicted, PBS treated or VRP-MPV.F vaccinated mice generated no detectable serum neutralizing titer. RSV-infected mice exhibited titers that peaked at day 28 post-infection and dropped gradually afterwards. VRP-RSV.F or VRP-RSV.G vaccination induced an increasing neutralizing titer after the first immunization, which peaked at 14 days after the first boost. Subsequent boosting did not enhance the level of neutralizing titer after the first boost, regardless of dosage (FIG. 5). Therefore, a single prime-boost was sufficient to generate effective neutralizing antibodies against RSV in vivo.

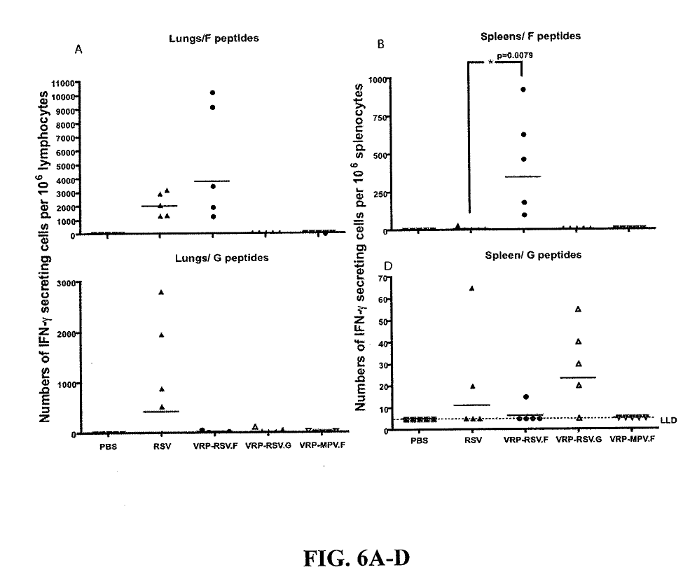

Cellular immunity in VRP-vaccinated mice. The inventors performed an IFN-γ ELISPOT assay to detect any RSV F- or G-specific T cells in the spleens or lungs of immunized animals. Lung lymphocytes and splenocytes were harvested separately 7 days after vaccination, stimulated in vitro with peptides representing known H-2^{d}-restricted RSV F (aa 85-93) or G (aa 183-197) CTL epitopes and the numbers of IFN-γ secreting cells were measured. The frequencies of RSV F specific CD4+/CD8+ T cells were higher in the VRP-RSV.F vaccinated group (ranging from 1,250-10,230 spots per 10⁶ lung lymphocytes) compared to the RSV-infected group (ranging from 1,285-3,180 spots per 10⁶ lung lymphocytes) (FIG. 6A). The frequency of RSV F-specific CD4+/CD8+ T cells in the lungs was 10-fold higher than that in the spleen (FIG. 6B). The responses of splenocytes or lung lymphocytes to RSV G epitopes were low. The frequencies of RSV G-specific CD4+/CD8+ T cells in RSV infected mice averaged 1,235 or 20 spots per 10⁶ lung lymphocytes or splenocytes respectively (FIGS. 6C and 6D). VRP-RSV.G vaccination induced limited responses in the spleen and no detectable CD4+/CD8+ T cells response in the lungs (FIGS. 6C and 6D), which is consistent with previous findings with SFV vaccination (Chen et al., 2002).

Viral titer in lungs and nasal turbinates after challenge in vaccinated mice. To assess the protective efficacy of VRP vaccines in vivo, the inventors measured the RSV titers in the lungs and nasal turbinates in mice and cotton rats following intranasal RSV challenge. Mice vaccinated with VRP-RSV.F were completely protected from RSV challenge at all dosage tested (35-fold or 47-fold reduction in lungs or nasal turbinates respectively). Previous infection with RSV also completely suppressed RSV growth in the upper and lower respiratory tracts. In contrast, mice vaccinated with VRP-RSV.G were protected from RSV challenge in the lungs but not in the nasal turbinates (Table 1). In the RSV permissive cotton rat model, vaccination with VRP-RSV.F protected both the upper and lower respiratory tracts of these animals (1000-fold or 25-fold reduction in the lungs or nasal turbinates) (Table 2).

TABLE 1
Titers of RSV in the lungs and nasal turbinates were reduced
in VRP-RSV.F vaccinated BALB/c mice after challenge

| | | | | Fold re- |
|---|---|---|---|---|
| | | RSV titer following challenge | | duction |
| | Dose* | (mean log₁₀pfu/g tissue ± SEM) | | of RSV |
| Immuni- | (log₁₀ | | Nasal | genomes^{†} |
| zation^{#} | PFU/IU) | Lungs | turbinates | Lungs |
| PBS | — | 3.25 ± 0.23 | 3.67 ± 0.23 | 1 |
| RSV | 6 | ≦1.7** | ≦2.0** | 23,042 |
| VRP-RSV.F | 4 | ≦1.7 | ≦2.0 | nd^{§} |
| | 5 | ≦1.7 | ≦2.0 | nd |
| | 6 | ≦1.7 | ≦2.0 | 12,077 |
| VRP-RSV.G | 4 | ≦1.7 | 3.00 ± 0.70 | nd |
| | 6 | ≦1.7 | 2.33 ± 0.85 | 204 |
| VRP-MPV.F | 6 | 3.03 ± 0.23 | 3.23 ± 0.25 | 3 |

*Titer of RSV [PFU] was determined by plaque formation in HEp-2 cells. Infection units [IU] of VRP were determined by number of infected BHK cells immunostained for VEE nonstructural proteins.
**Indicates virus was not detected at the limit of detection, 1.7 in the lungs or 2.0 in the nasal turbinates. Results are from groups of five animals.
^{#}Animals in each VRP group received 2 doses of VRPs while those in the RSV group were immunized once with RSV.
^{†}Fold differences were calculated based on the reduction of RSV genomes in the lungs 4 days after challenge compared to the amount of RSV genome in the lungs of PBS vaccinated animals.
^{§}Not determined

TABLE 2
RSV titers in the lungs and nasal turbinates were reduced
in VRP-RSV.F vaccinated cotton rats after challenge

| | | Serum neutral- | | |
|---|---|---|---|---|
| | | izing anti- | RSV titer following challenge | |
| | | body titer | (mean log₁₀pfu/g | |
| | Dose | at challenge | tissue ± SEM) | |
| Immuni- | (log₁₀ | (log₂ mean ± | | Nasal |
| zation^{#} | PFU/IU) | SEM) | Lungs | Turbinates |
| PBS | — | ≦4.32 | 4.0 ± 0.4 | 3.4 ± 0.5 |
| RSV | 6 | 7.4 ± 1.0 | ≦1.0* | ≦2.0* |
| VRP-RSV.F | 6 | 7.7 ± 0.8 | ≦1.0 | ≦2.0 |

*Indicates virus was not detected at the limit of detection, 1.0 in the lungs or 2.0 in the nasal turbinates.
^{#}Animals in each VRP group received 2 doses of VRPs while those in the RSV group were immunized once with RSV.

Histopathology and cytokine gene expression profile in VRP-vaccinated mice after RSV challenge. Lungs from VRP-vaccinated and control mice were removed on day 4 after RSV challenge and tested for histopathology and for cytokine gene expression. Lung sections were scored in a group-blinded fashion. In naïve mice challenged with RSV, there were mild mononuclear infiltrates in the alveolar space compared to uninfected controls. There was a moderate increase in mononuclear infiltrates in the alveolar, peribronchial and perivascular spaces of animals that were previously infected with RSV and in those that received VRP-RSV.F or VRP-RSV.G. The severity of inflammation was comparable between animals that were vaccinated with VRP-RSV.F and those previously infected with RSV. Animals vaccinated with VRP-RSV.G showed less inflammation. In contrast, mice vaccinated with formalin-inactivated RSV exhibited severe inflammation with alveolar inflammatory patches and abundant infiltration in the peribronchial and perivascular spaces. These animals also scored significantly higher than their VRP-vaccinated counterparts (Table 3). Mucus was not detected in any of the sections (data not shown).

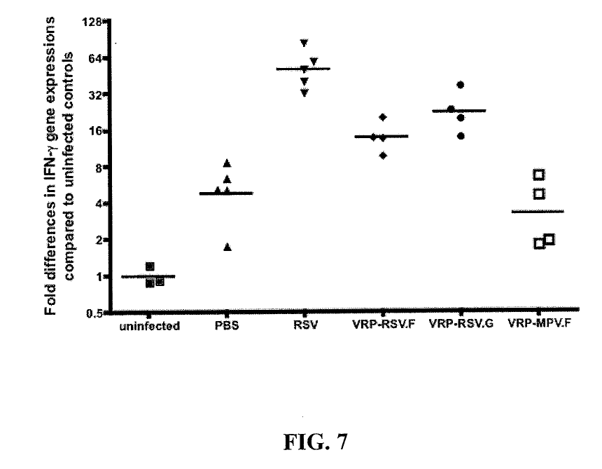

Cytokine gene expression levels were measured in the same tissues by reverse-transcribed real-time PCR on purified cellular RNA. Only IFN-γ gene expression in the lungs was upregulated in RSV challenged mice among all cytokines tested. None of the other cytokine genes tested (IL-2, IL-4, IL-5, IL-10 and IL-12) was statistically different when compared to uninfected controls (data not shown). Naïve animals and animals that received control replicons (VRP-MPV.F) had about 4-fold increase in IFN-γ gene transcription. Animals that were vaccinated with VRP or those previously infected with RSV had 16-50 fold increases in IFN-γ gene expression (FIG. 7).

TABLE 3
Histopathology scores of lung tissues in vaccinated
mice 4 days after wild-type RSV challenge

| | Histopathology score | | |
|---|---|---|---|
| Immuni- | Alveolar | Peribronchial | Perivascular |
| zation | tissue | tissue | tissue |
| Control | 0.2 ± 0.2 | 0.1 ± 0.1 | 0.1 ± 0.1 |
| RSV | 1.3 ± 0.4 | 1.3 ± 0.3 | 1.6 ± 0.2 |
| VRP-RSV.F | 1.1 ± 0.1 | 1.2 ± 0.2 | 1.7 ± 0.5 |
| VRP-RSV.G | 0.2 ± 0.2 | 0.8 ± 0.4 | 1.4 ± 0.3 |
| FI-RSV | 2.2 ± 0.2 | 2.2 ± 0.3 | 2.7 ± 0.1 |

Lung sections were viewed and scored by a pathologist in a group-blind fashion. Scores ranged from 0 (normal) to 3 or 4 (severe), as described in the method section.

### Example 3

### Materials & Methods

Animals and cell lines. 5-6 week old DBA/2 mice and cotton rats were purchased from Harlan (Indianapolis, Ind.) and Virion Systems (Rockville, Md.) respectively. Animals were housed in micro-isolator cages throughout the study. All experimental procedures performed were approved by the Institutional Animal Care and Use Committee at Vanderbilt University Medical Center.

LLC-MK2 cells were obtained from ATCC(CCL-7) and maintained in OptiMEM I medium (Invitrogen) supplemented with 2% fetal bovine serum (FBS), 4 mM L-glutamine, 5 μg/mL amphotericin B and 50 μg/mL gentamicin sulfate at 37° C. with 5% CO2. BHK-21 cells were obtained from ATCC(CCL-10) and maintained in Eagle's Minimum Essential Medium) supplemented with 10% fetal bovine serum (FBS), 4 mM L-glutamine, 5 μg/mL amphotericin B and 50 μg/mL gentamicin sulfate at 37° C. with VEE constructs and generation of VRPs encoding hMPV F or G genes. The method of construction and packaging of viral replicon particles (VRPs) was described previously (Pushko et al., 1997). Briefly, the hMPV fusion (F) or attachment (G) protein encoding DNA sequences from the subgroup A2 hMPV wild-type strain TN/94-49 were inserted behind the 26S subgenomic promoter in a VEE replicon plasmid, pVR21. pVR21 was derived from mutagenesis of a cDNA clone of the Trinidad donkey strain of VEE.

For generation of VRPs, capped RNA transcripts of the pVR21 plasmid containing hMPV F or G genes were generated in vitro with the mMESSAGE mMACHINE T7 kit (Ambion, Austin, Tex.). Similarly, helper transcripts that encoded the VEE capsid and glycoproteins genes derived from the attenuated recombinant V3014 strain were generated in vitro. BHK-21 cells then were co-transfected by electroporation with the pVR21 and helper RNAs and culture supernatants were harvested at 30 hours after transfection. The generation of VRPs expressing the F protein of the related virus RSV (used in the present studies as a heterologous virus control) was previously described (Mok et al., 2007).

VRP titration. Serial dilutions of VRPs encoding hMPV F (designated VRP-MPV.F) or hMPV G (designated VPR-MPV.G) were used to infect BHK cells in eight-chamber slides (Nunc) for 20 hours at 37° C. Infected BHK cells were fixed and immunostained for VEE non-structural proteins. Infectious units then were calculated from the number of VEE protein-stained cells per dilution and converted to infectious units (IU) per milliliter.

Formalin-inactivated hMPV (FI-hMPV) preparation. Sucrose gradient purified hMPV A2 (TN 94-49) strain was prepared as previously described (Williams et al., 2005b). Purified hMPV were inactivated with (1:4000 dilution) 37% formaldehyde solution for 72 hours at 37° C. The solution then was centrifuged at 50,000×g for an hour at 4° C. The resulting pellet was then resuspended 1:25 in serum-free optiMEM and precipitated with aluminum hydroxide (4 mg/ml) for 30 min. The precipitate was collected by centrifugation for 30 min at 1,000×g, resuspended 1:4 in serum-free optiMEM, and stored at 4° C. (44).

Immunofluorescence staining. BHK cells were infected at a moi of 5 with VRPMPV.F or VRP-MPV.G in eight-chamber slides (Nunc) for 18 hours at 37° C. Infected BHK cells were fixed in 80% methanol for an hour at 4° C. The cells then were blocked with PBS/3% BSA for two hours at room temperature. Monoclonal antibody against hMPV F or hMPV polyclonal guinea pig serum (1:1000 dilution in PBS/1% BSA) was added and allowed to incubate for an hour at room temperature. Cells were washed extensively with Tris-buffered saline/0.5% Tween-20 after incubation with primary antibodies, and secondary goat anti-mouse or goat anti-guinea pig AlexaFluor C568-conjugated antibodies were added (1:1000 dilution in TBST/1% BSA) to the cells for an additional hour. The slide then was washed with TBST and mounted with Prolong antifrade medium (Invitrogen, Carlsbad, Calif.). The slide was visualized using an LSM510 inverted laser scanning confocal microscope (Carl Zeiss Microimaging, Thornwood, N.Y.).

Vaccination and challenge of mice or cotton rats. DBA/2 mice were anesthetized with isoflurane and vaccinated intranasally with various titers of VRP-MPV.F or VRP-MPV.G in a 100 μL inoculum. Control groups were inoculated via the same route with phosphate buffered saline (PBS), 105.9 PFU of hMPV subgroup A2 wild-type strain TN/94-49, or 106 infectious units of VRPs encoding the RSV F gene (VRP-RSV.F). Mice that were vaccinated with VRPs were boosted with the same dose two weeks later. For histopathology and cytokine gene expression studies, a subgroup of animals was vaccinated once with 50 μl of FI-hMPV in each hind leg intramuscularly. The mice then were observed for clinical signs daily and bled on day 42 to follow immune responses.

Twenty-eight days after the second immunization (day 42), mice from VRP-MPV.F and VRP-MPV.G vaccinated groups and mice from the control groups were challenged with 105.9 PFU of the hMPV subgroup A2 strain TN/94-49 or subgroup B1 strain TN/98-242 intranasally. To monitor virus replication in the upper and lower respiratory tracts, nasal turbinates and lungs were harvested on day 4 post-challenge and subsequently assayed for virus titer. Similarly, cotton rats were vaccinated on day 0 and day 14 with 106 IU of VRP-MPV.F or VRP-MPV.G intranasally in groups of 4. Control groups were inoculated intranasally with PBS, 10⁵⁹ PFU of hMPV TN/94-49 or 10⁶ IU of VRP-RSV.F. They then were bled on day 35 to monitor immune responses, were challenged with 10^{5.9} PFU of hMPV TN/94-49 on day 42, and were sacrificed on day 46. Lung and nasal turbinates were harvested separately and homogenized to determine viral titers.

BAL fluid and nasal wash collection. A subset of animals was sacrificed on day 42 (28 days after the second immunization) to collect bronchoalveolar lavage fluid (BAL) and nasal wash fluid. BAL fluids were collected by ligation of the trachea with suture, insertion of a 23-gauge blunt needle into the distal trachea, followed by three in-and-out flushes of the airways with 3 mL of sterile PBS. Nasal washes were obtained by flushing 3 mL PBS through the upper trachea and out the nasal orifice into a sterile receptacle. Both BAL and nasal washes were concentrated 10-fold using 10 kD molecular weight cutoff Centricon concentrators (Millipore, Bedford, Mass.).

F protein and G protein-specific antibody assay. Sera collected at day 42 from DBA/2 mice were tested for the presence of F or G protein specific antibodies. Concentrated nasal washes and BAL fluids also were tested. Briefly, 150 ng/well of purified hMPV F protein or hMPV G protein was adsorbed onto Immulon 2B plates overnight in carbonate buffer (pH 9.8) at 4° C. Recombinant F protein was generated as described (13) and recombinant G protein was produced by similar methods (Ryder A B, Podsiad A B, Tollefson S J, Williams J V, unpublished data). The plates then were blocked with 3% bovine serum albumin (BSA) in PBS for 2 hours at room temperature. After thorough washing with TBST/1% BSA, serial dilutions of serum, nasal wash or BAL fluid samples were added to the plate and allowed to incubate for an hour at room temperature. The plates were washed again and horseradish peroxidase (HRP)-conjugated anti-mouse IgA (1:500 dilution) or IgG (1:5000 dilution) antibodies were added (Southern Biotech, Birmingham Ala.) and allowed to incubate for another hour. Finally, the plates were washed and 100 μL of One-Step Turbo TMB peroxidase substrate (Pierce, Rockford, Ill.) was added per well to quantify the relative amounts of F-specific or G-specific IgA or IgG in the samples. The reactions then were stopped by adding 50 μL of 1M HCl and the absorbance of the samples was read at 450 nm. The ELISA titers were expressed as the reciprocal titer of serum in which the absorbance was twice the background absorbance. Background absorbance was determined from the average OD₄₅₀ nm in PBS-incubated control wells.

Virus neutralizing antibody assay. Sera collected were used to study the presence of hMPV neutralizing antibodies as previously described (Williams et al., 2005b). Serum samples were tested for neutralizing activity against subgroup A1 strain TN/96-12, subgroup A2 strain TN/94-49, subgroup B1 strain TN/98-242 and subgroup B2 strain TN/99-419 of hMPV. Briefly, a viral suspension that was standardized to yield 50 plaques per well in a 24-well plate was used. An aliquot of the hMPV suspension was incubated with serial dilutions of the serum samples. After an hour, the suspension was absorbed onto LLC-MK2 cells and then overlaid an hour later with a semisolid methylcellulose overlay containing 5 μg/mL of trypsin. After 4 days, the cell culture monolayers were fixed and stained by immunoperoxidase using hMPV-specific polyclonal guinea pig serum to identify plaques. Plaques were counted and plaque reduction was calculated by regression analysis to provide a 60% plaque reduction titer.

Virus plaque titer assay. Serial dilutions of nasal turbinate or lung homogenates were inoculated onto LLC-MK2 cell monolayer cultures and plaque assays were performed as described above. Viral titer was determined by multiplying the number of plaques by reciprocal sample dilution, divided by tissue weights, and expressed as PFU/g tissue.

Lung histopathology studies. Four days after hMPV challenge, mice were euthanized with CO2 inhalation and lungs were harvested. To preserve structural integrity of the lungs, 1 mL of 10% neutral buffered formalin was instilled into the lungs via tracheotomy, followed by ligation of the trachea with sutures. The whole lung then was immersed in 10% neutral buffered formalin overnight. After fixation, the lungs were dehydrated by immersing in 70% ethanol for another day. The lungs then were embedded in paraffin, sectioned and stained with hematoxylin/eosin solution. The severity of airway inflammation was evaluated separately for the alveolar, peribronchial tissue and perivascular spaces in a group-blind fashion. The degree of inflammation in the alveolar tissue was graded as follows: 0, normal; 1, increased thickness of the interalveolar septa (IAS) by edema and cell infiltration; 2, luminal cell infiltration; 3, abundant cell infiltration; and 4, inflammatory patches were formed. The degree of inflammation in the peribronchial and perivascular spaces was graded as follows: 0, no infiltrate; 1, slight cell infiltration was noted; 2, moderate cell infiltration was noted; and 3, abundant cell infiltration was noted. In each tissue section, 10 alveolar tissue fields, 10 airways and 10 blood vessels were analyzed using 200× magnification. Mean scores were calculated for each mouse and an average score was reported for each animal group.

Cytokine gene expression in the lungs after hMPV challenge. Lungs from unvaccinated and vaccinated mice were harvested 4 days after hMPV challenge and placed in RNAlater solution (Ambion, Austin, Tex.) until further analysis. Lungs were homogenized using the Omni-tip PCR kit (Omni International, Marietta, Ga.) and RNA was extracted using the RNeasy Mini kit (Qiagen, Valencia, Calif.) according to the manufacturer's protocol. Primers and probes for real time quantitative PCR were purchased from Applied Biosystems (Foster City, Calif.) to measure Th1 or Th2 cytokine transcript levels based on GenBank sequences for murine GAPDH, gamma interferon (IFN-γ) and interleukins 2 (IL-2), 4 (IL-4), 5 (IL-5), 10 (IL-10) and 12 (IL-12). Probes were labeled at the 5′ end with 6-carboxyfluorescein (FAM) and at the 3′ end with the nonfluorescent quencher Blackhole Quencher 1 (BHQ1; Operon Biotechnologies, Huntsville, Ala.). Reverse-transcribed real-time PCR was performed using Quantitect Probe RT-PCR kit (Qiagen, Valencia, Calif.) and a Smart Cycler II (Cepheid, Sunnyvale, Calif.) using 1 μg of extracted mRNA. The parameters used were 1 cycle of 50° C. for 2 min, 1 cycle of 95° C. for 10 min, and 40 cycles of 95° C. for 15 sec and 60° C. for 1 min. Reactions were performed in triplicate, with a no-template sample used as a negative control. Relative amounts of cytokine gene transcripts expressed were normalized to those of the GAPDH housekeeping gene, and uninfected mice were used as baseline controls. Differences in mRNA levels were computed using the DDCt method, comparing to uninfected mice.

Statistics. Prism software was used to plot the data (Graphpad Software Inc., San Diego, Calif.). All data were expressed as geometric means and their standard deviations. Data also were analyzed by Mann-Whitney rank sum test to compare the sample means between any two experimental groups using Prism.

### Example 4

### Results

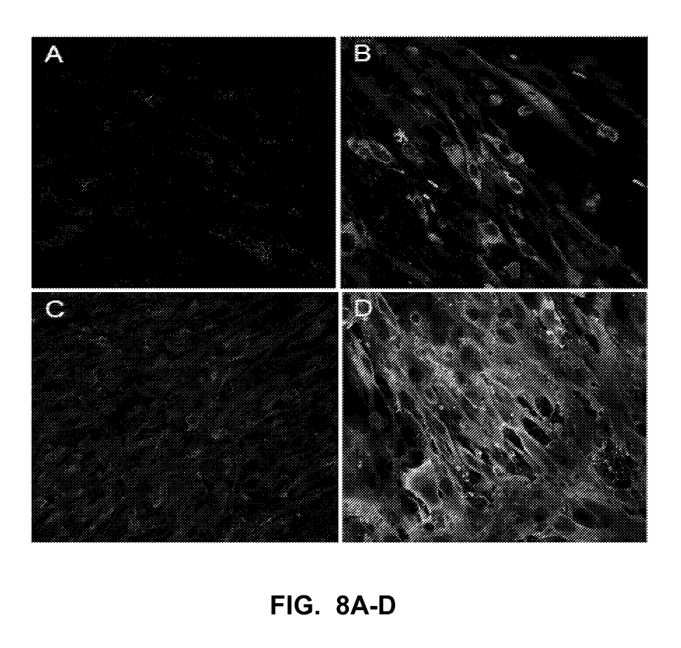

Cloning and expression of hMPV antigens using VEE replicon particles (VRPs). hMPV fusion (MPV.F) and attachment (MPV.G) genes were cloned into the VEE replicon vector as previously described (Pushko et al., 1997). VRPs then were produced in BHK cells by cotransfecting RNA transcribed in vitro from the replicon vector with transcripts of two separate plasmids encoding VEE capsid and envelope proteins in trans. To ensure these replicons expressed the desired antigens, BHK cells were infected at a moi of 5 with VRPs. Antigen expression then was measured by immunostaining infected cells with guinea pig polyclonal hMPV-specific antibodies. A robust amount of hMPV F or G protein was expressed, as evident from the intense staining of infected BHK cells with hMPV-specific antibodies (FIGS. 8B and 8D), compared to uninfected cells (FIGS. 8A and 8C). Examination of infected cells by confocal microscopy showed a Golgi and membrane-bound expression pattern for hMPV F protein, while staining of cells with MPV.G VRP showed a membrane-bound pattern. Western blots also were used to confirm the presence of hMPV F or G protein expression in BHK-infected cell lysates (data not shown).

Systemic IgG and mucosal IgA responses in VRP-vaccinated mice. To assess if VRPs could induce systemic humoral immune responses, the inventors measured the reciprocal endpoint titers of hMPV F- or G-specific IgG antibodies in the serum of vaccinated mice by ELISA. Intranasal inoculation of hMPV F-VRPs induced significantly higher titers of hMPV F-specific IgG in the sera of vaccinated mice (about 8-fold higher in both the 10⁶ and 10⁵ IU groups) than in unvaccinated animals. These animals possessed 2-fold higher antibody titer compared to mice infected once with hMPV, a difference that did not reach statistical significance (p=0.22). Similarly, mice that were vaccinated with VRP-MPV.G showed robust levels of hMPV G-specific IgG in the sera (298-fold and 20-fold higher in 10⁶ and 10⁵ IU groups respectively) compared to unvaccinated control animals (Table 4).

TABLE 4
Serum antibody responses against hMPV F
and G proteins in immunized DBA/2 mice

| | Dose | Serum reciprocal endpoint ELISA titer | |
|---|---|---|---|
| Immuni- | (log₁₀ IU | (mean log₂ titer ± SD^{#}) against | |
| zation | or PFU) | hMPV-F | hMPV G |
| PBS | — | 9.6 ± 0.5 | 4.4 ± 0.2 |
| VRP-RSV.F | 6 | 9.8 ± 0.5 | ≦4.3 |
| VRP-MPV.F | 6 | 12.9 ± 1.5** | 4.6 ± 0.6 |
| | 5 | 12.8 ± 1.7* | nd |
| | 4 | 10.4 ± 0.8 | nd |
| VRP-MPV.G | 6 | 9.8 ± 0.4 | 12.3 ± 1.1** |
| | 5 | nd^{†} | 8.7 ± 1.5** |
| | 4 | nd | 7.3 ± 2.1 |
| hMPV | 5.9 | 11.8 ± 1.0** | 5.0 ± 1.5 |

^{†}Not determined
^{#}Statistical significance of serum reciprocal endpoint ELISA titer when compared to PBS-vaccinated group:
*p < 0.05
**p < 0.01

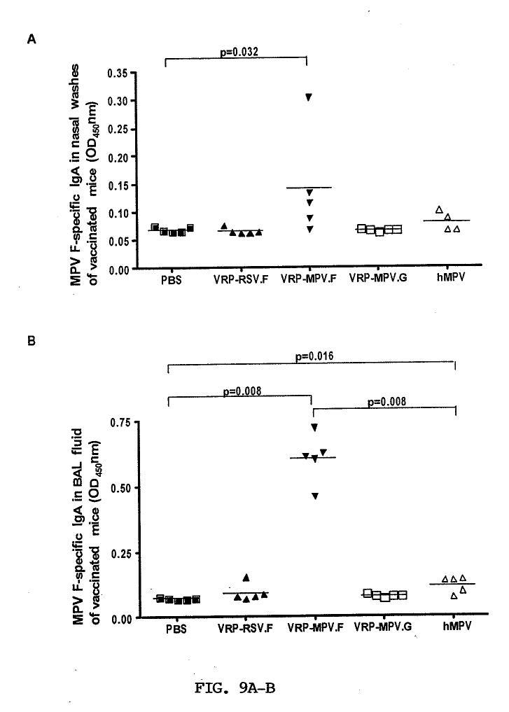

Mucosal hMPV F-specific or G-specific IgA antibodies also were detected in the nasal washes and bronchioalveolar lavage (BAL) fluids of VRP-MPV.F or VRP-MPV.G vaccinated mice respectively, which represent the presence of immunity in the upper or lower respiratory tracts of vaccinated animals (FIGS. 9A and 9B). Significantly higher titers of hMPV F-specific or hMPV G-specific antibodies were observed in the BAL fluids of VRP-MPV.F or VRP-MPV.G vaccinated mice compared to hMPV-infected mice (p=0.008), possibly due to repeated exposures to antigens during priming and boosting of the VRP-vaccinated animals. Alternatively, the higher anti-F and anti-G BAL antibody titers could also be due to presentation of the viral antigens from a different target cell in the case of VRP vaccination.

Neutralizing activity of antibodies in the sera of VRP-vaccinated animals. The presence of circulating neutralizing antibodies is an important parameter that has been implicated to protect the lower respiratory tract against respiratory virus infection, including against hMPV. Therefore, the inventors measured neutralizing activity in the sera from VRP-vaccinated mice or cotton rats against subgroup A or B hMPV strains using a 60% plaque reduction assay. Mice vaccinated with PBS or VRP expressing RSV F protein, used as a heterologous virus control, did not generate any detectable neutralizing titer against either subgroup A or B hMPV strains. Intranasal vaccination with VRP-MPV.F induced at least a 2.3 log₂ (5-fold) or 1.8 log₂ (3.5-fold) increase in serum neutralizing antibody titer against the A2 or A1 subgroup of hMPV when compared to PBS-vaccinated mice (Table 5). Neutralizing activity against subgroup A2 strain MPV was higher than against subgroup A1 strain in these animals. When these sera were tested against subgroup B hMPV in our 60% plaque reduction assay in vitro, all sera tested had minimal neutralizing activity towards subgroup B hMPV. There is some neutralizing activity at the lowest serum dilution 1:20, which however did not reach our 60% plaque reduction criteria in two separate experiments (Table 5). Surprisingly, infection with subgroup A2 hMPV did not induce serum antibodies that could neutralize subgroup B viruses in vitro. Neutralizing titers also were not detected in mice vaccinated with VRP-MPV.G, despite the presence of hMPV G-specific IgG in these animals (Table 4). Mice that were infected with a subgroup A2 and A1 strains of hMPV respectively, but very little neutralizing activity against subgroup B hMPV.

In cotton rats, a similar trend was observed for neutralizing activity against subgroup A hMPV. Intranasal vaccination with 106 IU of VRP-MPV.F induced reciprocal neutralizing titers of 6.7 log₂ and 5.7 log₂ against subgroup A2 and A1 strains of hMPV, compared to 9.6 log₂ and 6.0 log₂ from hMPV-infected animals (Table 5). The neutralization responses were higher in cotton rats than mice when immunized with hMPV, likely because the cotton rat is a more permissive model for hMPV infection as evidenced by higher viral titers in the nasal turbinates of these animals (Table 6).

TABLE 5
Serum neutralizing antibody responses against various
hMPV strains in immunized DBA/2 mice or cotton rats

| | Dose | 60% Plaque reduction sreum neutraliing titer (mean log₂ titer ± SD) against hMPV | | | | | | | |
|---|---|---|---|---|---|---|---|---|---|
| | (log₁₀ IU | DBA/2Mice | | | | Cotton Rats | | | |
| Immunization | or PFU) | A1^{#} | A2^{#} | B1^{#} | B2^{#} | A1^{#} | A2^{#} | B1^{#} | B2^{#} |
| PBS | — | ≦4.3* | ≦4.3 | ≦4.3 | ≦4.3 | ≦4.3 | ≦4.3 | ≦4.3 | ≦4.3 |
| VRP-RSV.F | 6.0 | ≦4.3 | ≦4.3 | ≦4.3 | ≦4.3 | ≦4.3 | ≦4.3 | ≦4.3 | ≦4.3 |
| VRP-MPV.F | 6.0 | 6.1 ± 1.7 | 6.6 ± 1.9 | ≦4.3 | ≦4.3 | 5.7 ± 1.2 | 6.7 ± 2.3 | ≦4.3 | ≦4.3 |
| VRP-MPV.G | 6.0 | ≦4.3 | ≦4.3 | ≦4.3 | ≦4.3 | ≦4.3 | ≦4.3 | ≦4.3 | ≦4.3 |
| MPV A2 | 5.9 | 6.3 ± 1.2 | 7.7 ± 1.3 | ≦4.3 | ≦4.3 | 6.0 ± 0.6 | 9.6 ± 0.9 | ≦4.3 | ≦4.3 |

*Lower limit of detection was 4.3 log₂ for hMPV neutralization titer
^{#}The hMPV subgroup A1 strain was TN/96-12; the subgroup A2 strain was TN/94-49; the subgroup B1 strain was TN/98-242 and the subgroup B2 strain was TN/99-419

TABLE 6
hMPV titers in the lungs or nasal turbinates of immunized DBA/2 mice
or cotton rats following wild-type subgroup A2 or B1 hMPV challenge

| | MPV titer following challenge (mean log₁₀ pfu/g tissue ± SD | | | | | |
|---|---|---|---|---|---|---|
| | DBA/2 (A2) | | DBA/2 (B1) | | Cotton Rats (A2) | |
| Immunization | Lungs | Nasal Turbinates | Lungs | Nasal Turbinates | Lungs | Nasal Turbinates |
| PBS | 3.9 ± 0.4 | 3.5 ± 0.2 | 3.5 ± 0.3 | 3.5 ± 0.3 | 3.4 ± 0.8 | 4.5 ± 0.4 |
| VRP-RSV.F | 3.4 ± 0.2 | 3.4 ± 0.1 | 3.3 ± 0.5 | 3.8 ± 0.2 | 4.2 ± 0.0 | 4.5 ± 0.6 |
| VRP-MPV.F | ≦1.7^{#} | 2.5 ± 0.5^{††} | ≦1.7^{#} | 3.0 ± 0.3 | ≦1.5* | 2.2 ± 0.5^{†} |
| VRP-MPV.G | 3.0 ± 0.7 | 3.0 ± 0.3 | 3.6 ± 0.2 | 3.4 ± 0.4 | 3.5 ± 0.3 | 4.6 ± 0.3 |
| MPV A2 | ≦1.7^{#} | ≦2.0^{#} | ≦1.7^{#} | 2.2 ± 0.3^{§} | ≦1.5* | ≦2.0* |

Designation in parenthesis indicates the subgroup of hMPV used for challenge.
^{#}Lower limit of detection was 1.7 log₁₀ or 2.0 log₁₀ for the lungs or nasal turbinates of DBA/2 mice respectively.
*Lower limit of detection was 1.5 log₁₀ or 2.0 log₁₀ for the lungs or nasal turbinates of cotton rats respectively.
^{††}2 out of 5 mice had an undetectable hMPV A2 titer in the nasal turbinates.
^{§}2 out of 5 mice had an undetectable hMPV B1 titer in the nasal turbinates.
^{†}3 out of 4 cotton rats had an undetectable hMPV A2 titer in the nasal turinates.

Viral titer in lungs and nasal turbinates after challenge in vaccinated animals. To assess the protective efficacy of VRP vaccines in vivo, the inventors measured hMPV titers in the lungs or nasal turbinates of mice or cotton rats following intranasal hMPV subgroup A2 challenge. Mice or cotton rats vaccinated with VRP-MPV.F had no detectable challenge hMPV titers in the lungs (at least a 2.2 log_{10 [}158-fold] or 1.9 log_{10 [}79-fold] reduction in mice or cotton rats respectively). Reduced amounts of hMPV also were observed in the nasal turbinates of VRP-MPV.F vaccinated animals (1.0 log_{10 [}10-fold] or 2.3 log_{10 [}200-fold] reduction in mice or cotton rats, respectively). Previous infection with hMPV subgroup A2 induced immunity resulting in a reduction of hMPV challenge titers to undetectable levels in both the upper and lower respiratory tracts. In contrast, mice or cotton rats vaccinated with VRP-MPV.G were not protected from hMPV challenge in either the lungs or nasal turbinates (Table 6), which is consistent with the lack of serum neutralizing antibodies the inventors observed. In addition, the inventors challenged their vaccinated mice with a subgroup B1 strain hMPV. In the lungs of VRP-MPV.F vaccinated mice, viral titers were reduced 1.8 log₁₀ (63-fold) when compared with the PBS-vaccinated group. This surprising reduction was possibly due to the presence of low level of neutralizing antibodies in these animals. In a semi-permissive mouse model, a low amount of neutralizing antibodies may be sufficient to reduce hMPV replication in the lower respiratory tract. In animals previously infected with a MPV subgroup A2 strain, the inventors observed a similar magnitude of viral titer reduction in the lungs when challenged with a subgroup B1 strain virus.

Histopathology of the lungs after challenge in vaccinated animals. The inventors evaluated the extent of cellular infiltrates in the perivascular, peribronchial and alveolar spaces in the lungs of mice vaccinated with VRP and then challenged with wild-type hMPV. In animals that received mock PBS vaccination, a minimal amount of infiltration was observed 4 days post-hMPV infection. In animals that were previously infected with hMPV, re-infection of mice with hMPV caused a dramatic increase in cellular infiltrates in the perivascular, peribronchial and alveolar spaces of the lungs.

There was also a moderate increase in mononuclear infiltrates in the alveolar, peribronchial and perivascular spaces of animals that received VRP-MPV.F or VRPMPV.G when challenged with wild-type hMPV. The histopathology scores were comparable and not statistically different between animals that were vaccinated with VRP-MPV.F and those previously infected with hMPV when both groups were challenged with wild-type hMPV, although mice vaccinated with VRP-MPV.F did show a trend of decreased severity of inflammation in the peribronchial and perivascular tissues upon challenge. In contrast, animals that were vaccinated with a single dose of formalin-inactivated hMPV and challenged with wild-type virus exhibited extensive cell infiltrations in the perivascular, peribronchial and alveolar spaces, which are evidenced by the increased histopathology scores when compared to other vaccination groups (Table 7). This phenomenon is consistent with previous findings (Yim et al., 2007).

TABLE 7
Histopathology scores of lung tissues in vaccinated
mice 4 days after wild-type MPV challenge

| | Histopathology score | | |
|---|---|---|---|
| Immuni- | Alveolar | Peribronchial | Perivascular |
| zation | tissue | tissue | tissue |
| PBS | 0.4 ± 0.4 | 0.1 ± 0.1 | 0.2 ± 0.1 |
| MPV | 0.8 ± 0.2 | 0.9 ± 0.2 | 1.2 ± 0.1 |
| VRP-MPV.F | 1.0 ± 0.3 | 0.6 ± 0.2 | 0.7 ± 0.3 |
| VRP-MPV.G | 0.8 ± 0.5 | 0.3 ± 0.1 | 0.4 ± 0.2 |
| VRP-RSV.F | 0.7 ± 0.2 | 0.5 ± 0.4 | 0.4 ± 0.3 |
| FI-MPV | 1.4 ± 0.2 | 1.1 ± 0.2 | 1.8 ± 0.5 |

Lung sections viewed and scored by pathologist in a group-blind fashion. Scores ranged from 0 (normal) to 3 or 4 (severe), as described in the Methods section.

Cytokine mRNA expression in lungs of vaccinated mice after challenge. Aberrant cytokine responses and enhanced disease after subsequent natural exposure have been observed in animals or humans vaccinated with certain non-replicating paramyxovirus vaccines. Recently, formalin-inactivated hMPV has been shown to induce a Th2-biased cytokine response and aggravated disease in experimental animals (Yim et al., 2007). The inventors measured cytokine mRNA levels in the lungs of VRP-vaccinated mice after hMPV challenge to investigate if VRP vaccines would cause such biased responses. For each of the cytokine mRNAs tested, hMPV-infected mice had increased lung cytokine mRNA levels over uninfected controls. The mRNA expression levels of IFN-γ, IL-4, IL-10, IL-12p40 or IL-13 were not statistically different between groups, with 2 exceptions. There was a 2.6-fold reduction of IFN-γ gene expression in the lungs of VRP-MPV.F vaccinated mice compared to PBS controls and a 2.1-fold increase in IL-10 gene expression in the lungs of VRP-MPV.G vaccinated mice compared to PBS controls. As predicted, in formalin-inactivated hMPV vaccinated animals, there is statistically significant decrease in IFN-γ and IL-12p40 mRNA and a statistically significant increase in IL-13 compared to PBS controls (Table 8).

TABLE 8
Cytokine mRNA expression in the lungs of immunized
DBA/2 mice following wild-type subgroup A2 hMPV

| | Mean fold difference in cytokine gene expression comparied to uninfected controls (range^{#}) | | | | |
|---|---|---|---|---|---|
| Immunization | IFN-γ | IL-4 | IL-10 | IL-12 p40 | IL-13 |
| PBS | 8.3 (4.6-17.9) | 2.2 (1.3-5.1) | 3.7 (2.6-7.0) | 9.3 (6.2-14.2) | 15.3 (7.4-46.6) |
| VRP-RSV.F | 5.4 (4.1-8.6) | 1.8 (1.4-3.2) | 3.9 (2.5-5.2) | 9.7 (3.7-16.9) | 11.6 (4.2-24.0) |
| VRP-MPV.F | 3.2 (2.2-4.9)* | 2.2 (1.1-3.7) | 4.4 (2.1-6.7) | 15.5 (10.9-23.9) | 10.8 (6.7-14.5) |
| VRP-MPV.G | 10.8 (6.3-19.5) | 1.9 (1.1-2.9) | 7.8 (4.7-10.0)* | 12.3 (6.3-19.8) | 15.8 (6.3-24.5) |
| MPV | 8.3 (4.0-10.5) | 2.1 (1.4-3.6) | 4.9 (2.3-9.3) | 14.8 (9.0-21.9) | 6.0 (3.3-13.9) |
| FI-MPV | 3.0 (2.1-6.8)* | 4.0 (2.1-8.1) | 2.9 (1.5-7.4) | 4.7 (2.3-7.5) | 82.7 (27-208)* |

^{#}Values in parentheses indicate the range of fold differences between 5 mice in each group
*Statistical significance of group was detected when compared to PBS vaccinated group, p < 0.05 (Mann-Whitney Test)

Immunogenicity in the presence of passively-acquired antibodies. The target population for RSV and MPV vaccination, young infants, possess RSV- and MPV-specific neutralizing antibodies of maternal origin that are transplacentally acquired. Such antibodies are suppressive of immune responses to conventional vaccines that possess RSV or MPV antigens on their surface. One of the benefits of the present invention is that the replicon particles making up the vaccine matter do not display RSV or MPV antigens on the surface, and thus are not bound by antibodies to these antigens. Also, in contrast to other vectors such poxviruses, adenoviruses, and other common viral vectors, most humans do not possess antibodies to the VEE vector or replicon proteins. Thus, the VEE replicons should escape the suppressive effects of passively-acquired RSV- or MPV-specific antibodies. Laboratory experiments in mice have proven this to be true. First, the inventors prepared mouse immune serum by infection mice with RSV, and then collected the serum. Passive transfer of the immune serum to naïve mice, followed by RSV replicon immunization or wild-type RSV infection, showed that the immune response to RSV, but not to the replicon vaccine, was suppressed.

Multiple modes of immune protection. The inventors also performed experiments to define the mechanism by which the replicons induced immunity. Interestingly, they found that the vaccine constructs induce both humoral and cell-mediated immune elements that contribute to immunity. First, the inventors immunized mice with replicon vaccines, then collected immune serum and transferred that serum to naïve mice. The antibody-treated mice were protected from infection, showing that antibodies induced by VEE vectored RSV vaccine are sufficient to mediate protection. Next, the inventors immunized μMT mice, which lack B cells. Vaccination in these mice also induced protection, suggest that something other than B cells and antibodies can contribute to protection. The inventors performed T cell assays including interferon-γ ELISPOTS and flow cytometric assays with defined RSV BALB/c F protein T cell epitopes, and showed that vaccination with the replicons induced T cells that mediated protection in the absence of antibodies.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. References

The following references, to the extent that they provide exemplary procedural or other details supplementary to those set forth herein, are specifically incorporated herein by reference.
U.S. Pat. No. 5,185,440
U.S. Pat. No. 5,505,947
U.S. Pat. No. 5,643,576
U.S. Pat. No. 5,792,462
U.S. Pat. No. 6,156,558
U.S. Pat. No. 6,521,235
U.S. Pat. No. 6,531,135
U.S. Pat. No. 6,541,010
U.S. Pat. No. 6,738,939
U.S. Pat. No. 7,045,335
U.S. Pat. No. 7,078,218
U.S. Patent Pubn. 2002/014975 A1
Balasuriya et al., Vaccine, 20(11-12):1609-17, 2002.
Bastien et al., J. Clin. Microbiol., 42:3532-7, 2004.
Bastien et al., Virus Res., 93:51-62, 2003.
Biacchesi et al., J. Virol., 78:12877-87, 2004.
Biacchesi et al., Virology, 315:1-9, 2003.
Boivin et al., Emerg. Infect. Dis., 10:1154-7, 2004.
Boivin et al., J. Infect. Dis., 186:1330-4, 2002.
Bukreyev et al., J. Virol., 80:5854-61, 2006.
Burkhard et al., Vaccine, 21(3-4):258-68, 2002.
Cannon et al., J. Exp. Med., 168:1163-8, 1988.
Chen et al., J. Immunol., 169(6):3208-16, 2002.
Connors et al., J. Virol., 65(3):1634-7, 1991.
Connors et al., J. Virol., 66(2):1277-81, 1992.
Connors et al., Virology, 208(2):478-84, 1995.
Corvaisier et al., Res. Virol., 144(2):141-50, 1993.
Crowe et al., J. Infect. Dis., 173(4):829-39, 1996a.
Crowe et al., Virus Genes, 13(3):269-73, 1996b.
Crowe et al., J. Infect. Dis., 177(4):1073-6, 1998.
Crowe et al., Vaccine, 11(14):1395-404, 1993.
Crowe et al., Vaccine, 12(9):783-90, 1994a.
Crowe et al., Vaccine, 12(8):691-9, 1994b.
Crowe et al., Vaccine, 13(9):847-55, 1995.
Cseke et al., J. Virol., 81:698-707, 2007.
Davis et al., IUBMB Life, 53(4-5):209-11, 2002.
Davis et al., J. Virol., 70(6):3781-7, 1996.
Durbin et al., J. Immunol., 168(6):2944-52, 2002.
Ebihara et al., J. Clin. Microbiol., 42:5944-6, 2004.
Esper et al., J. Infect. Dis., 189:1388-96, 2004.
Falsey et al., J. Infect. Dis., 187:785-90, 2003.
Firestone et al., Virology, 225(2):419-22, 1996.
Gingras et al., Proc. Natl. Acad. Sci. USA, 93:5578 5583, 1996.
Gipson et al., Vaccine, 21(25-26):3875-84, 2003.
Gradi et al., Proc. Natl. Acad. Sci. USA, 95:11089 11094, 1998.
Haller et al., J. Gen. Virol., 84(Pt 8):2153-62, 2003.
Hancock et al., J. Virol., 70(11):7783-91, 1996.
Harrington et al., J. Virol., 76(2):730-42, 2002.
Herd et al. J. Virol., 80:2034-44, 2006.
Hershey & Merrick, In: Translational Control of Gene Expression, 33-88, Cold Spring Harbor Laboratory Press, NY, 2000.
Hevey et al., Virology, 251(1):28-37, 1998.
Homa et al., J. Gen. Virol., 74(9):1995-9, 1993.
Hsu et al., J. Infect. Dis., 166(4):769-75, 1992.
Hsu et al., Vaccine, 13(5):509-15, 1995.
Huckriede et al., Vaccine, 22(9-10):1104-13, 2004.
Jackson, In: Translational Control of Gene Expression, 127-184, Cold Spring Harbor Laboratory Press, NY, 2000.
Joachims et al., J. Virol., 73:718 727, 1999.
Johnson et al., J. Virol., 72(4):2871-80, 1998.
Johnston et al., Vaccine, 23(42):4969-79, 2005.
Juhasz et al., J. Virol., 71(8):5814-9, 1997.
Kahn et al., J. Virol., 75(22):11079-87, 2001.
Kapikian et al., Am. J. Epidemiol., 89(4):405-21, 1969.
Karron et al., J. Infect. Dis., 176(6):1428-36, 1997.
Karron et al., J. Infect. Dis., 191(7):1093-104, 2005.
Kim et al., Am. J. Epidemiol., 89(4):422-34, 1969.
Kulkarni et al., J. Virol., 67(2):1044-9, 1993.
Lee et al., J. Infect. Dis., 185(8):1192-6, 2002.
MacDonald et al., J. Virol., 74(2):914-22, 2000.
Macejak and Sarnow, Nature, 353:90-94, 1991.
Martinez-Salas et al., J. Gen. Virol., 82:973 984, 2001.
Martinez-Sobrido et al., J. Virol., 80(3):1130-9, 2006.
McAdam et al., J. Infect. Dis., 190:20-6, 2004.
Melendi et al., J. Virol., 81(20):11461-7, 2007.
Moghaddam et al., Nat. Med., 12(8):905-7, 2006.
Mok et al., J. Virol., 81(24):13710-22, 2007.
Moran et al. J. Immunol., 175(5):3431-8, 2005.
Murphy et al., J. Clin. Microbiol., 24(2):197-202, 1986.
Murphy et al., J. Virol., 62(10):3907-10, 1988.
Olmsted et al., Proc. Natl. Acad. Sci. USA, 83(19):7462-6, 1986.
Osterhaus and Fouchier, Lancet., 361:890-1, 2003.
Peebles et al., J Allergy Clin. Immunol., 113(1):S15-8, 2004.
Pelletier et al., Emerg. Infect. Dis., 8:976-8, 2002.
Peroulis et al., Arch. Virol., 144(1):107-16, 1999.
Plotnicky-Gilquin et al., J. Virol., 76:10203-10, 2002.
Polack et al. Proc. Natl. Acad. Sci. USA, 102:8996-9001, 2005.
Power et al., Virology, 230(2):155-66, 1997.
Prince et al. J. Virol., 55:517-20, 1985.
Prince et al., J. Virol., 57(3):721-8, 1986.
Prince et al., Virus Res., 3(3):193-206, 1985.
Pushko et al., J. Virol., 75(23):11677-85, 2001.
Pushko et al., Virology, 239(2):389-401, 1997.
Remington's Pharmaceutical Sciences, 18th Ed. Mack Printing Company, pp. 1289-1329, 1990.
Ryman et al., J. Virol., 79(3):1487-99, 2005.
Sami et al., J. Infect. Dis., 171(2):440-3, 1995.
Schultz-Cherry et al., Virology, 278(1):55-9, 2000.
Skiadopoulos et al., J. Virol., 78:6927-37, 2004.
Skiadopoulos et al., Virology, 345:492-501, 2006.
Takimoto et al., J. Virol., 78(11):6043-7, 2004.
Tang et al., J. Virol., 77:10819-28, 2003.
Tang et al., Vaccine, 23:1657-67, 2005.
Tebbey et al., J. Exp. Med., 188(10):1967-72, 1998.
Teng et al., Virology, 289(2):283-96, 2001.
Thompson & Sarnow, Curr. Opin. Microbio., 3:366 370, 2000.
Thompson et al., Proc. Natl. Acad. Sci. USA, 103(10):3722-7, 2006.
Tripp et al., Nat. Immunol., 2:732-8, 2001.
van den Hoogen et al., Nat. Med., 7:719-24, 2001
Varga et al., Immunity, 15:637-46, 2001.
Vaux-Peretz et al., Vaccine, 8(6):543-8, 1990.
Velders et al., Cancer Res., 61(21):7861-7, 2001.
Walsh, Vaccine, 11(11):1135-8, 1993.
Wang et al., Breast Cancer Res., 7(1):R145-55, 2005.
Waris et al., J. Virol., 70(5):2852-60, 1996.
Welliver et al., J. Infect. Dis., 170(2):425-8, 1994.
White et al., J. Virol., 75(8):3706-18, 2001.
Williams et al. N. Engl. J. Med., 350:443-50, 2004.
Williams et al., J. Infect. Dis., 192:1061-5, 2005.
Williams et al., J. Infect. Dis., 192:1149-53, 2005.
Williamson et al., AIDS Res. Hum. Retroviruses, 19(2):133-44, 2003.
Wyatt et al., Vaccine, 18(5-6):392-7, 1999.
Yang and Sarnow, Nucleic Acids Res., 25:2800-7, 1997.
Yim et al., Vaccine, 25:5034-40, 2007.
You et al., Respir. Res., 7:107, 2006.

What is claimed is:

1. A virus replicon comprising:
(a) a Venezuelan equine encephalitis virus (VEE) positive-sense RNA genome lacking at least one functional gene for an VEE structural gene; and
(b) a paramyxovirus surface glycoprotein coding region under the control of a promoter active in eukaryotic cells.

2. The replicon of claim 1, wherein said paramyoxovirus surface glycoprotein coding region is from respiratory syncytial virus.

3. The replicon of claim 2, wherein said RSV glyprotein coding region is RSV F or G.

4. The replicon on claim 1, wherein said paramyoxovirus surface glycoprotein coding region is from human metapneumovirus (hMPV).

5. The replicon of claim 4, wherein said hMPV glyprotein coding region is hMPV F.

6. The replicon of claim 1, wherein said promoter is the VEE subgenomic 26S promoter.

7. The replicon of claim 1, wherein said VEE RNA genome is from pVR21.

8. The replicon of claim 1, wherein said VEE RNA genome contains an inactivating point mutation in a structural gene.

9. The replicon of claim 1, wherein said VEE RNA genome contains a truncating mutation in a structural gene.

10. The replicon of claim 1, wherein said VEE RNA genome contains a deletion mutation in a structural gene.

11. A method of inducing an immune response in an animal comprising administering to said animal an infectious virus particle comprising a viral replicon comprising:
(a) a Venezuelan equine encephalitis virus (VEE) positive-sense RNA genome lacking at least one functional gene for an VEE structural gene; and
(b) a paramyxovirus surface glycoprotein coding region under the control of a promoter active in eukaryotic cells.

12. The method of claim 11, wherein said paramyoxovirus surface glycoprotein coding region is from respiratory syncytial virus.

13. The method of claim 12, wherein said RSV glycprotein coding region is RSV F or G.

14. The method on claim 11, wherein said paramyoxovirus surface glycoprotein coding region is from human metapneumovirus (hMPV).

15. The method of claim 14, wherein said hMPV glyprotein coding region is hMPV F.

16. The method of claim 11, wherein said promoter is the VEE subgenomic 26S promoter.

17. The method of claim 11, wherein said VEE RNA genome is from pVR21.

18. The method of claim 11, wherein said VEE RNA genome contains an inactivating point mutation in a structural gene.

19. The method of claim 11, wherein said VEE RNA genome contains a truncating mutation in a structural gene.

20. The method of claim 11, wherein said VEE RNA genome contains a deletion mutation in a structural gene.

21. The method of claim 11, wherein said animal is a human.

22. The method of claim 21, wherein said human is a neonate comprising maternal antibodies.

23. The method of claim 11, wherein said animal is a mouse.

24. The method of claim 11, wherein administration comprises intranasal inhalation, subcutaneous injection or intramuscular injection.

25. The method of claim 11, further comprising administering said infectious virus particle a second time.

26. The method of claim 11, further comprising administering said infectious virus particle a third time.

27. The method of claim 11, further comprising assessing an immune response to said paramyxovirus surface glycoprotein.

28. The method of claim 26, wherein assessing comprises RIA, ELISA, immunohistochemistry or Western blot.

29. The method of claim 1, wherein said immune response is a humoral response.

30. The method of claim 29, wherein said humoral response is mucosal IgA.

31. The method of claim 29, wherein said humoral response is serum IgG.

32. The method of claim 31, wherein said serum IgG response is neutralizing.

33. The method of claim 1, wherein said immune response is cellular.

34. The method of claim 33, wherein said cellular response is a balanced Th1/Th2 response.
